(19) United States
(12) Patent Application Publication
(10) Pub. No.: US 2024/0369545 A1
(43) Pub. Date: Nov. 7, 2024

(54) MULTIPLEXED PROTEOMICS ANALYSIS USING OLIGONUCLEOTIDE-CONJUGATED ANTIBODIES

(71) Applicant: Becton, Dickinson and Company, Franklin Lakes, NJ (US)

(72) Inventors: Adam Thomas Wright, San Jose, CA (US); Jody Martin, San Jose, CA (US); Hye-Won Song, San Jose, CA (US); Samatha Vadrevu, San Jose, CA (US)

(21) Appl. No.: 18/686,824

(22) PCT Filed: Aug. 30, 2022
(86) PCT No.: PCT/US2022/075655

§ 371 Date: Feb. 26, 2024

Related U.S. Application Data
(60) Provisional application No. 63/239,379, filed on Aug. 31, 2021.

(51) Int. Cl. G01N 33/53; G01N 33/543; G01N 33/58; G01N 33/68

(52) U.S. Cl. CPC G01N 33/5308; G01N 33/54313; G01N 33/582; G01N 33/588; G01N 33/6854

(57) ABSTRACT

Antibodies attached to oligonucleotides can bind to proteins of cells. These oligonucleotides can be released and captured by solid support oligonucleotides attached to solid supports. A detection oligonucleotide can hybridize to the captured oligonucleotides forming a sandwich of solid support oligonucleotide-captured oligonucleotide-detection oligonucleotide. The detection oligonucleotide and the captured oligonucleotides it binds to can be quantified by, for example, the fluorescent intensity of second detectable moieties (e.g., dye(s)) on the detection oligonucleotide. The identity of the protein quantified can be determined by, for example, the fluorescent intensity of first detectable moieties (e.g., dye(s)) on the solid support with the sandwich of solid support oligonucleotide-captured oligonucleotide-detection oligonucleotide.

### RELATED APPLICATIONS

This application is a U.S. national phase application under 35 U.S.C. § 371 of International Application No. PCT/US2022/075655, filed on Aug. 30, 2022, which claims the benefit under 35 U.S.C. § 119 (e) of U.S. Provisional Patent Application No. 63/239,379, filed on Aug. 31, 2021. The content of these related applications is incorporated herein by reference in its entirety for all purposes.

### BACKGROUND

### Field

The present disclosure relates generally to the field of molecular biology, for example performing multiplexed proteomics analysis using oligonucleotide-conjugated antibodies.

### Description of the Related Art

Multiplexed proteomics allows for the quantification of many proteins from various samples. Current multiplexed antibody-based assays require proteins that are readily released into solution and antibody pairs to identify protein targets of interest. There is a need for multiplexed assays that can quantify proteins not readily released into solution and do not require antibody pairs to identify protein targets of interest.

### SUMMARY

Disclosed herein include methods of cellular component quantification. In some embodiments, the method comprises: contacting one or more cells of each of a plurality of samples with a plurality of cellular component binding reagents each associated with a reagent oligonucleotide, wherein two of the plurality of cellular component binding reagents are capable of binding to two different cellular components, or two different regions of a cellular component, and wherein each reagent oligonucleotide comprises (i) a reagent-specific sequence specific to the cellular component binding reagent associated thereto and (ii) a detection sequence, to obtain cells comprising cellular components bound to cellular component binding reagents of the plurality of cellular component binding reagents. The method can comprise: removing cellular component binding reagents of the plurality of cellular component binding reagents not bound to the cells. The method can comprise: contacting reagent oligonucleotides associated with cellular component binding reagents of the plurality of cellular component binding reagents not removed with a plurality of solid supports, wherein each of the plurality of solid supports is associated with one or more first detectable moieties, or precursors thereof, and comprises a plurality of solid support oligonucleotides, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences, to obtain reagent oligonucleotides bound to the plurality of solid supports. The method can comprise: contacting the reagent oligonucleotides bound to the plurality of solid supports with a detection oligonucleotide associated with one or more second detectable moieties, or precursors thereof and comprising a binding sequence capable of binding to the detection sequences of the reagent oligonucleotides to obtain reagent oligonucleotides bound to the plurality of solid supports and the detection oligonucleotide. The method can comprise: detecting the one or more first detectable moieties and the one or more second detectable moieties for each of the plurality of solid supports to determine the identity and the quantity, respectively, of each of the cellular components for each of the plurality of samples.

Disclosed herein include methods of cellular component quantification. The method can comprise: (a) providing one or more cells from each of a plurality of samples and with cellular components bound to cellular component binding reagents of a plurality of cellular component binding reagents (1) capable of binding to different cellular components, or regions thereof, and (2) each associated with a reagent oligonucleotide comprising (i) a reagent-specific sequence specific to the cellular component binding reagent associated thereto, and (ii) a detection sequence; and The method can comprise for each of the plurality of samples: (b) contacting reagent oligonucleotides, associated with or previously associated with the cellular component binding reagents bound to the cellular components of or from the cells of the sample, with a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of the reagent oligonucleotides, thereby obtaining reagent oligonucleotides bound to the plurality of solid supports; (c) contacting the reagent oligonucleotides bound to the plurality of solid supports with a detection oligonucleotide associated with one or more second detectable moieties, or precursors thereof, and comprising a binding sequence capable of binding to the detection sequences of the reagent oligonucleotides, thereby obtaining reagent oligonucleotides bound to both the plurality of solid supports and the detection oligonucleotide; and (d) detecting the one or more first detectable moieties and the one or more second detectable moieties for each of the plurality of solid supports to determine the identity and the quantity, respectively, of each of the cellular components for each of the plurality of samples.

In some embodiments, determining the identity and the quantity of each of the cellular components for each of the plurality of samples comprises: detecting the presence and/or amount of the one or more first detectable moieties and the one or more second detectable moieties for each of the plurality of solid supports. In some embodiments, the presence and/or amount of the one or more first detectable moieties and the presence and/or amount of the one or more second detectable moieties determined for a solid support indicate the identity and the quantity, respectively, of each of the cellular components for each of the plurality of samples. In some embodiments, detecting the presence and/or amount of the one or more first detectable moieties and the one or more second detectable moieties comprises: measuring emissions of the one or more first detectable moieties and the one or more second detectable moieties with an instrument, optionally measuring emissions using flow cytometry (e.g., fluorescence-activated cell sorting (FACS)).

In some embodiments, one or more of the first detectable moieties and/or the second detectable moieties comprise an optical moiety, a luminescent moiety, an electrochemically active moiety, a nanoparticle, or a combination thereof. In some embodiments, the luminescent moiety comprises a chemiluminescent moiety, an electroluminescent moiety, a photoluminescent moiety, or a combination thereof. In some embodiments, the photoluminescent moiety comprises a fluorescent moiety, a phosphorescent moiety, or a combination thereof. In some embodiments, the fluorescent moiety comprises a fluorescent dye. In some embodiments, the nanoparticle comprises a quantum dot. The method can comprise: performing a reaction to convert the detectable moiety precursor into the detectable moiety. The method can comprise: (i) contacting two or more solid supports with two or more predetermined concentrations of a cellular component binding reagent, wherein each of the two or more solid supports is contacted with a different predetermined concentration of the cellular component binding reagent; (ii) contacting the two or more solid supports with the reagent oligonucleotides; and (iii) measuring emissions of the one or more second detectable moieties of each of the two or more first solid supports with an instrument to generate a calibration curve relating the quantity of at least one cellular component to emissions of the one or more second detectable moieties. In some embodiments, the instrument comprises a flow cytometer. In some embodiments, the flow cytometer comprises a conventional flow cytometer, a spectral flow cytometer, a hyperspectral flow cytometer, an imaging flow cytometer, or any combination thereof.

In some embodiments, contacting one or more cells of each of a plurality of samples with a plurality of cellular component binding reagents comprises: partitioning the plurality of samples to a plurality of partitions, wherein a partition of the plurality of partitions comprises a single sample of the plurality of samples; and contacting one or more cells of each of a plurality of samples with a plurality of cellular component binding reagents. In some embodiments, contacting one or more cells of each of a plurality of samples with a plurality of cellular component binding reagents comprises: contacting one or more cells of each of a plurality of samples with a plurality of cellular component binding reagents; and partitioning the plurality of samples to a plurality of partitions, wherein a partition of the plurality of partitions comprises a single sample of the plurality of samples. In some embodiments, providing one or more cells from each of a plurality of samples and with cellular components bound to cellular component binding reagents of a plurality of cellular component binding reagents comprises: providing a plurality of partitions each comprising a sample of the plurality of samples, wherein a partition of the plurality of partitions comprises a single sample of the plurality of samples. In some embodiments, the plurality of samples are partitioned to the plurality of partitions prior to contacting the one or more cells of each of a plurality of samples with a plurality of cellular component binding reagents, wherein a partition of the plurality of partitions comprises a single sample of the plurality of samples. In some embodiments, the partition is a well or a droplet. In some embodiments, the plurality of partitions comprises wells of a well array. In some embodiments, the well array comprises at least about 10 to 100 wells.

In some embodiments, the instrument comprises a fluorescence microscope. In some embodiments, the instrument comprises an imaging system. In some embodiments, measuring emissions of each detectable moiety of each first solid support comprises imaging the plurality of partitions. In some embodiments, the plurality of partitions are imaged sequentially. In some embodiments, the plurality of partitions are imaged simultaneously. In some embodiments, imaging comprises microscopy, confocal microscopy, time-lapse imaging microscopy, fluorescence microscopy, multi-photon microscopy, quantitative phase microscopy, surface enhanced Raman spectroscopy, videography, manual visual analysis, automated visual analysis, or any combination thereof.

In some embodiments, detecting the one or more first detectable moieties and the one or more second detectable moieties for each of the plurality of solid supports comprises: detecting the one or more first detectable moieties and the one or more second detectable moieties for each of the plurality of solid supports of each sample of the plurality of samples separately, thereby determining the identity and the quantity, respectively, of each of the cellular components for each sample of the plurality of samples. In some embodiments, detecting the one or more first detectable moieties and the one or more second detectable moieties for each of the plurality of solid supports of each sample of the plurality of samples separately comprises detecting the one or more first detectable moieties and the one or more second detectable moieties for each of the plurality of solid supports of each partition separately. In some embodiments, the one or more first detectable moieties of the plurality of solid supports situated in each partition are predetermined, and said predetermined one or more first detectable moieties are distinct to each partition. In some embodiments, detecting the one or more first detectable moieties and the one or more second detectable moieties for each of the plurality of solid supports comprises: detecting the predetermined one or more first detectable moieties and the one or more second detectable moieties for each of the plurality of solid supports of each sample of the plurality of samples concurrently; and associating the detected predetermined one or more first detectable moieties of each of the solid supports with the partition from which said solid support derived, thereby determining the identity and the quantity, respectively, of each of the cellular components for each sample of the plurality of samples. The method can comprise: pooling the solid supports from each partition of the plurality of partitions (e.g., using a magnetic field). In some embodiments, contacting the reagent oligonucleotides bound to the plurality of solid supports with the detection oligonucleotide comprises: contacting the reagent oligonucleotides bound to the plurality of solid supports with two or more detection oligonucleotides each associated with one or more second detectable moieties.

In some embodiments, the two or more detection oligonucleotides are associated with an identical second detectable moieties. In some embodiments, the two or more detection oligonucleotides are associated with different second detectable moieties. In some embodiments, the two or more detection oligonucleotides comprise an identical binding sequence. In some embodiments, the two or more detection oligonucleotides comprise different binding sequences. In some embodiments, each of the plurality of solid supports is associated with two distinct first detectable moieties. In some embodiments, two solid supports of the plurality of solid supports comprise different types and/or quantities of the two distinct first detectable moieties. The method can comprise: isolating one or more populations of interest from a starting population to obtain the plurality of samples. In some embodiments, each of the samples is a population of interest. In some embodiments, two or more of the samples of the plurality of samples comprise phenotypically different populations of interest. In some embodiments, isolating one or more populations of interest from a starting population comprises flow cytometry (e.g., fluorescence-activated cell sorting (FACS)). In some embodiments, providing the cells comprises: contacting the cells of each of the plurality of samples with the plurality of cellular component binding reagents to obtain the cells with the cellular components bound to the cellular component binding reagents. In some embodiments, providing the cells comprises: removing cellular component binding reagents of the plurality of cellular component binding reagents not bound to the cells to obtain the cells with the cellular components bound to the cellular component binding reagents. In some embodiments, removing the cellular component binding reagents not bound to the cells comprises washing the cells with a washing buffer. The method can comprise: permeabilizing and/or fixating the cells prior to contacting the cells of each of the plurality of samples with the plurality of cellular component binding reagents. In some embodiments, two of the plurality of cellular component binding reagents are capable of binding to two different cellular components. In some embodiments, two of the plurality of cellular component binding reagents are capable of binding to two different regions of a cellular component. The method can comprise: isolating the one or more cells from the sample. In some embodiments, isolating the one or more cells comprises isolating the one or more cells from the sample using flow cytometry (e.g., fluorescence-activated cell sorting (FACS)). The method can comprise: lysing the cells, prior to contacting the reagent oligonucleotides with the plurality of solid supports. The method can comprise: dissociating the reagent oligonucleotides from the cellular component binding reagents bound to or previously bound to the cellular components of or from the cells of the sample, prior to contacting the reagent oligonucleotides with the plurality of solid supports. In some embodiments, dissociating the reagent oligonucleotides comprises: detaching the reagent oligonucleotides from the cellular component binding reagents bound to or previously bound to the cellular components of or from the cells of the sample by UV photocleaving, chemical treatment, heat treatment, enzyme treatment, or a combination thereof.

In some embodiments, the cellular components comprise a protein, a lipid, a carbohydrate, or a combination thereof. In some embodiments, the cellular components comprise an extracellular cellular component, a cell surface cellular component, an intracellular cellular component, or a combination thereof. In some embodiments, the plurality of cellular component binding reagents comprises a protein, an antibody, an aptamer, a tetramer, a protein scaffold, or a combination thereof. In some embodiments, the aptamer and the reagent oligonucleotide is a single polynucleotide. In some embodiments, the plurality of cellular component binding reagents comprises at least 10 cellular component binding reagents. In some embodiments, the reagent oligonucleotide is attached, releasably attached, covalently attached, non-covalently attached, and/or conjugated to the cellular component binding reagent. In some embodiments, the reagent oligonucleotide is associated with the cellular component through a UV photocleavable group and/or a chemical labile group. In some embodiments, the reagent oligonucleotide is associated with the cellular component through a linker. In some embodiments, the linker comprises a carbon chain. In some embodiments, the carbon chain comprises 2-30 carbons (e.g., 12 carbons) In some embodiments, the linker comprises 5′ amino modifier C12 (5AmMC12), or a derivative thereof. In some embodiments, the reagent oligonucleotide is 10 to 500 nucleotides in length. In some embodiments, the reagent-specific sequence is 5 to 495 nucleotides in length. In some embodiments, the detection sequence is 5 to 495 nucleotides in length. In some embodiments, one or more of the reagent oligonucleotides each comprises two or more reagent-specific sequences and/or two or more detection sequences, and/or wherein one or more of the reagent oligonucleotides each has a hairpin structure. In some embodiments, the reagent oligonucleotides comprise an identical detection sequence. In some embodiments, two of the reagent oligonucleotides comprise different detection sequences.

The method can comprise: amplifying the reagent oligonucleotides associated with or previously associated with the cellular component binding reagents bound to the cellular components of or from the cells of the sample to obtain amplified reagent oligonucleotides. In some embodiments, contacting the reagent oligonucleotides, associated with or previously associated with the cellular component binding reagents bound to the cellular components of or from the cells of the sample, comprises: contacting the amplified reagent oligonucleotides with the plurality of solid supports, thereby obtaining amplified reagent oligonucleotides bound to the plurality of solid supports. In some embodiments, contacting the reagent oligonucleotides bound to the plurality of solid supports comprises: contacting the amplified reagent oligonucleotides bound to the plurality of solid supports with the detection oligonucleotide, thereby obtaining amplified reagent oligonucleotides bound to both the plurality of solid supports and the detection oligonucleotide.

In some embodiments, at least two solid support oligonucleotides of a solid support of the plurality of solid supports comprises an identical capture sequence for binding to one of the reagent-specific sequences. In some embodiments, a solid support oligonucleotide of a first solid support and a solid support oligonucleotide of a second solid support of the plurality of solid supports comprise different capture sequences for binding to two different reagent-specific sequences of the reagent-specific sequences. In some embodiments, two solid supports of the plurality of solid supports comprise different quantities of the one or more first detectable moieties. In some embodiments, two solid supports of the plurality of solid supports comprise different first detectable moieties. In some embodiments, all solid supports of the plurality of solid supports are distinguishable from each other by the presence and/or amount of the one or more first detectable moieties associated thereto. In some embodiments, the one or more first detectable moieties is attached, releasably attached, covalently attached, non-covalently attached, and/or conjugated to the solid support.

In some embodiments, the plurality of solid supports comprises at least 10 solid supports. In some embodiments, the solid support comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the solid support comprises a material selected from polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. In some embodiments, each of the plurality of the solid support oligonucleotides is 10 to 500 nucleotides in length. In some embodiments, the capture sequence of each of the plurality of solid support oligonucleotides is 10 to 500 nucleotides in length. In some embodiments, the detection oligonucleotide is 10 to 500 nucleotides in length. In some embodiments, the binding sequence is 10 to 500 nucleotides in length. In some embodiments, the one or more second detectable moieties is attached, releasably attached, covalently attached, non-covalently attached, and/or conjugated to the detection oligonucleotide.

Disclosed herein include kits. In some embodiments, the kit comprises: a plurality of cellular component binding reagents (1) capable of binding to different cellular components, or regions thereof, and (2) each associated with a reagent oligonucleotide comprising (i) a reagent-specific sequence specific to a cellular component binding reagent associated with the reagent oligonucleotide, and (ii) a detection sequence. The kit can comprise: a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of reagent oligonucleotides. The method can comprise: a detection oligonucleotide associated with one or more second detectable moieties, or precursors thereof, and comprising a binding sequence capable of binding to the detection sequences of the reagent oligonucleotides.

### BRIEF DESCRIPTION OF THE DRAWINGS

FIG. 1 shows a schematic illustration of a non-limiting exemplary method of protein quantification.

FIG. 2 shows a schematic illustration of an exemplary protein binding reagent (antibody illustrated here) associated with an oligonucleotide comprising a unique identifier for the protein binding reagent.

FIG. 3A shows a non-limiting exemplary plot (a two-dimensional array) of intensities of two bead dyes, which can be used to determine the identity of the bead and the cellular component. FIG. 3B shows a schematic illustration of determining the identity of the cellular component using the intensities of two bead dyes (left hand side) and the quantifying the cellular component using the intensity of the detection dye (right hand side).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Multiplexed proteomics allows for the quantification of many proteins from various samples. Current multiplexed antibody-based assays require proteins that are readily released into solution and antibody pairs to identify protein targets of interest. There is a need for multiplexed assays that can quantify proteins (or other cellular components such as lipids and carbohydrates) not readily released into solution and do not require antibody pairs to identify protein targets (or other cellular components) of interest.

Disclosed herein include methods of cellular component quantification. In some embodiments, the method comprises: contacting one or more cells of each of a plurality of samples with a plurality of cellular component binding reagents each associated with a reagent oligonucleotide, wherein two of the plurality of cellular component binding reagents are capable of binding to two different cellular components, or two different regions of a cellular component, and wherein each reagent oligonucleotide comprises (i) a reagent-specific sequence specific to the cellular component binding reagent associated thereto and (ii) a detection sequence, to obtain cells comprising cellular components bound to cellular component binding reagents of the plurality of cellular component binding reagents. The method can comprise: removing cellular component binding reagents of the plurality of cellular component binding reagents not bound to the cells. The method can comprise: contacting reagent oligonucleotides associated with cellular component binding reagents of the plurality of cellular component binding reagents not removed with a plurality of solid supports, wherein each of the plurality of solid supports is associated with one or more first detectable moieties, or precursors thereof, and comprises a plurality of solid support oligonucleotides, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences, to obtain reagent oligonucleotides bound to the plurality of solid supports. The method can comprise: contacting the reagent oligonucleotides bound to the plurality of solid supports with a detection oligonucleotide associated with one or more second detectable moieties, or precursors thereof and comprising a binding sequence capable of binding to the detection sequences of the reagent oligonucleotides to obtain reagent oligonucleotides bound to the plurality of solid supports and the detection oligonucleotide. The method can comprise: detecting the one or more first detectable moieties and the one or more second detectable moieties for each of the plurality of solid supports to determine the identity and the quantity, respectively, of each of the cellular components for each of the plurality of samples.

Disclosed herein include methods of cellular component quantification. In some embodiments, the method comprises: (a) providing one or more cells from each of a plurality of samples and with cellular components bound to cellular component binding reagents of a plurality of cellular component binding reagents (1) capable of binding to different cellular components, or regions thereof, and (2) each associated with a reagent oligonucleotide comprising (i) a reagent-specific sequence specific to the cellular component binding reagent associated thereto, and (ii) a detection sequence; and The method can comprise for each of the plurality of samples: (b) contacting reagent oligonucleotides, associated with or previously associated with the cellular component binding reagents bound to the cellular components of or from the cells of the sample, with a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of the reagent oligonucleotides, thereby obtaining reagent oligonucleotides bound to the plurality of solid supports; (c) contacting the reagent oligonucleotides bound to the plurality of solid supports with a detection oligonucleotide associated with one or more second detectable moieties, or precursors thereof, and comprising a binding sequence capable of binding to the detection sequences of the reagent oligonucleotides, thereby obtaining reagent oligonucleotides bound to both the plurality of solid supports and the detection oligonucleotide; and (d) detecting the one or more first detectable moieties and the one or more second detectable moieties for each of the plurality of solid supports to determine the identity and the quantity, respectively, of each of the cellular components for each of the plurality of samples.

Disclosed herein include kits. In some embodiments, the kit comprises: a plurality of cellular component binding reagents (1) capable of binding to different cellular components, or regions thereof, and (2) each associated with a reagent oligonucleotide comprising (i) a reagent-specific sequence specific to a cellular component binding reagent associated with the reagent oligonucleotide, and (ii) a detection sequence. The kit can comprise: a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of reagent oligonucleotides. The method can comprise: a detection oligonucleotide associated with one or more second detectable moieties, or precursors thereof, and comprising a binding sequence capable of binding to the detection sequences of the reagent oligonucleotides.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, an antibody can be a full-length (e.g., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

In some embodiments, an antibody is a functional antibody fragment. For example, an antibody fragment can be a portion of an antibody such as F(ab′)2, Fab′, Fab, Fv, sFv and the like. An antibody fragment can bind with the same antigen that is recognized by the full-length antibody. An antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (for example, CD8, CD34, and CD45), and therapeutic antibodies.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one).

As used herein, the term “solid support” can refer to discrete solid or semi-solid surfaces to which a plurality of barcodes (e.g., stochastic barcodes) may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.”

### Proteomics Analysis

Multiplexed proteomics allows for the quantification of many proteins from various samples. In particular, the ability to analyze complex cell samples is of keen interest for biomarker discovery. Current techniques are limited in sensitivity, cost efficiency, and number of targets that can be analyzed. Antibodies provide a highly specific and sensitive route to study proteins present in biological samples. Current multiplexed antibody-based assays are limited to proteins readily released into solution and are burdened with non-trivial development of antibody pairs to identify targets of interest.

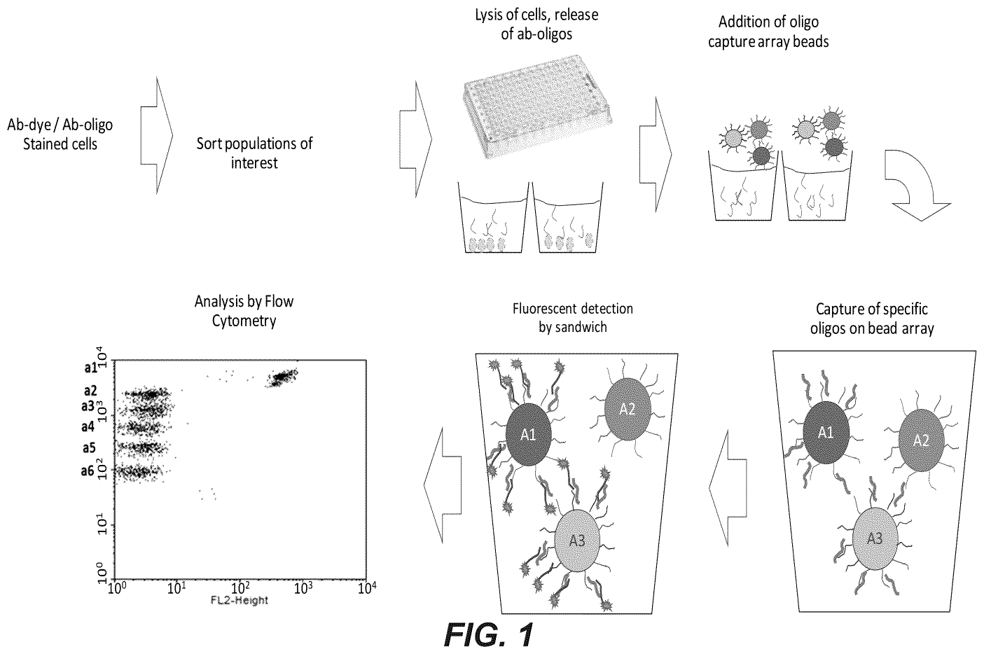

The methods and kits disclosed herein enable highly multiplexed protein expression analysis from a cell sample without the need for protein being released into solution or protein capture/detection via paired antibody probes. The methods and kits can enable a simple route to highly multiplexed, sensitive and quantitative protein analysis. Referring to FIG. 1, cellular proteins in their native state can be targeted using antibodies associated with (e.g., conjugated with) barcodes oligonucleotides. An oligonucleotide-conjugated with an antibody or an oligonucleotide previously conjugated with an antibody can be referred to herein as an antibody oligonucleotide (abbreviated as an “AbOligo” or “AbO”). Cell lysis can be followed by barcode analysis. First, cells can be stained with antibodies associated with oligonucleotides. The cell sample may be purified using any method known in the art or split into immuno-phenotyped subpopulations via, for example, FACS. Labeled cell populations of interest can be lysed as a bulk population to release the barcode oligonucleotides that quantitatively correlate to antibody bound proteins of interest. At this point, a simple and straight-forward sandwich assay can be applied to the barcode oligonucleotides from each sample in parallel. The barcode oligonucleotides can contain two key sequences, the unique antibody clone specific sequence as well as a universal detection sequence. These sequences can be spaced, duplicated, or hair-pinned for performance optimization. A multiplexed, fluorescently addressed, and oligonucleotide-coated bead array can be used to capture the barcode oligonucleotides. The bead oligonucleotides can each be addressed to a specific fluorescent position as in a bead array. Capture of barcode oligonucleotides may occur with hybridization complements covalently bound to beads such that each fluorescently addressed bead captures a specific antibody clone's oligonucleotide. After washing of unbound oligonucleotides and other cell sample material, detection of bead captured oligos can occur. Detection may be accomplished using hybridization of a fluorophore conjugated complement to the generic detection region of the captured barcode oligonucleotides. This simple sandwich assay can be accomplished with minimal expertise and acquired on a variety of fluorescent platforms including flow cytometry. Protein expression data is thus converted into a synthetic nucleic acid barcode. This enables easy sandwich, capture and detection, assays. The antibody oligonucleotides can also serve as a robust and releasable label in the presence of cell lysis or other perturbations. Determining the identity and the quantity of each of the cellular components for each of the plurality of samples can comprises detecting the presence and/or amount of the one or more first detectable moieties (e.g., bead dye(s)) and the one or more second detectable moieties (e.g., detection dye(s)) for each of the plurality of solid supports (e.g., beads). A user can associate the presence and/or amount of the one or more first detectable moieties with the species of reagent oligonucleotide bound to the solid support (and thereby associate the species of cellular component bound by the cellular component binding reagent). A user can detect the amount of the one or more second detectable moieties associated with each solid support to determine the quantity of each cellular component. The method can comprise analysis of a plurality of samples, each comprising one or more cells. The method can comprise quantifying one or more cellular components for each sample of the plurality of samples. In some embodiments, the solid supports of each of the samples is analyzed separately (e.g., each sample is in a separate partition, and the detectable moieties associated with solid supports each partition is detected separately). The solid supports of each of the samples can be analyzed in parallel (e.g., each sample is in a separate partition, distinct first detectable moieties of each partition are predetermined, solid supports are pooled and detectable concurrently).

Alternatively or additionally, the barcode oligonucleotides could be amplified prior to analysis, instead of directly analyzed. This can enable very sensitive analysis, down to the single cell or molecule level. In some embodiments, proteomics data generated using the methods disclosed herein (e.g., highly multiplexed population proteomics) can be bioinformatically coupled to the sorting (e.g., indexed FACS) data.

Currently, bulk proteomics is largely accomplished via mass spectrometry, which is insensitive and lacks specificity. Bulk analysis of proteins via antibody-based arrays is limited in the types of proteins targeted as well as manufacturability due to the fact that functional antibody pairs is necessary for most platforms. Key advantages of the methods and kits of the present disclosure include the ability to target any antibody accessible on or inside of cells via antibody labeling, sensitivity, specificity, and limitless multiplexing.

Existing methods such as Cytometric Bead Array and Luminex type assays can only be used to quantify proteins in a solution. In comparison, the methods and kits described herein can enable the analysis of any protein addressable with an antibody on or in intact cells. Additionally, the synthetic and uniform nature of the barcode oligonucleotides can enable downstream multiplexed analysis via any variety of oligonucleotide array. In addition to multiparameter protein analysis, other cellular components can be analyzed using cellular component binding reagents coupled to barcode oligonucleotides.

Disclosed herein include methods of protein quantification. In some embodiments, a method of protein quantification comprises: contacting one or more cells of a sample with a plurality of antibodies, each associated with an antibody oligonucleotide, to obtain cells comprising proteins bound to the plurality of antibodies. Each of the plurality of antibodies is capable of binding to a different protein. Each antibody oligonucleotide can comprise (i) an antibody sequence specific to the antibody associated with the antibody oligonucleotide and (ii) a detection sequence. The method can comprise: lysing the cells. The method can comprise: dissociating the antibody oligonucleotides from the plurality of antibodies of the lysed cells. The method can comprise: contacting the dissociated antibody oligonucleotides with a plurality of beads (or solid supports or particles) to obtain antibody oligonucleotides bound to the plurality of beads. Each of the plurality of beads is associated with a bead dye. Each of the plurality of solid supports (e.g., beads) can comprise a plurality of solid support oligonucleotides (e.g., bead oligonucleotides). The plurality of bead oligonucleotides of a bead of the plurality of beads can comprise an identical capture sequence for binding to one of the antibody sequences. The pluralities of bead oligonucleotides of two beads of the plurality of beads can comprise different capture sequences for binding to two different antibody sequences of the antibody sequences. The method can comprise: contacting the antibody oligonucleotides bound to the plurality of beads with a detection oligonucleotide to obtain antibody oligonucleotides bound to the plurality of beads and the detection oligonucleotide. The detection oligonucleotide is associated with a detection dye. The detection oligonucleotide can comprise a binding sequence capable of binding to the detection sequences of the antibody oligonucleotides. The method can comprise: determining an intensity of the bead dye and an intensity of the detection dye for each of the plurality of beads. The method can comprise: determining a quantity of each of the proteins based on the intensity of the bead dye and the intensity of the detection dye determined for each of the plurality of beads.

Disclosed herein include methods of protein quantification. In some embodiments, a method of protein quantification comprises: contacting one or more cells of a sample with a plurality of protein binding reagents, each associated with a reagent oligonucleotide, to obtain cells comprising proteins bound to the plurality of protein binding reagents. Two of the plurality of protein binding reagents can be capable of binding to two different proteins, or two different epitopes of a protein. Each reagent oligonucleotide can comprise a reagent-specific sequence specific to the protein binding reagent associated with the reagent oligonucleotide. Each reagent oligonucleotide can comprise a detection sequence. The method can comprise: dissociating the reagent oligonucleotides from the plurality of protein binding reagents bound to (or previously bound to) the cells. The method can comprise: contacting the dissociated reagent oligonucleotides with a plurality of beads (or particles) to obtain reagent oligonucleotides bound to the plurality of beads. Each of the plurality of beads can be associated with a bead dye. Each of the plurality of beads can comprise a plurality of bead oligonucleotides. At least two bead oligonucleotides of the plurality of bead oligonucleotides of a bead of the plurality of beads can comprise an identical capture sequence for binding to one of the reagent-specific sequences. A bead oligonucleotide of a first plurality of bead oligonucleotides of a first bead of the plurality of beads and a bead oligonucleotide of a second plurality of bead oligonucleotides of a second bead of the plurality of beads can comprise different capture sequences for binding to two different reagent-specific sequences of the reagent-specific sequences. The method can comprise: contacting the reagent oligonucleotides bound to the plurality of beads with a detection oligonucleotide to obtain reagent oligonucleotides bound to the plurality of beads and the detection oligonucleotide. The detection oligonucleotide can be associated with a detection dye. The detection oligonucleotide can comprise a binding sequence capable of binding to the detection sequences of the reagent oligonucleotides. The method can comprise: determining an intensity of the bead dye and an intensity of the detection dye for each of the plurality of beads. The method can comprise: determining a quantity of each of the proteins based on the intensity of the bead dye and the intensity of the detection dye determined for each of the plurality of beads.

### Cellular Component Analysis

Disclosed herein include methods of cellular component (e.g., a protein) quantification. In some embodiments, a method of cellular component quantification comprises: (a) providing one or more cells from each of a plurality of sample and with cellular components bound to cellular component binding reagents of a plurality of cellular component binding reagents. The plurality of cellular component binding reagents is capable of binding to different cellular components, or regions thereof. Each of the plurality of cellular component binding reagents is associated with a reagent oligonucleotide. The reagent oligonucleotide comprises a reagent-specific sequence specific to the cellular component binding reagent the reagent oligonucleotide is associated with. The reagent oligonucleotide can comprise a detection sequence. In some embodiments, two of the plurality of cellular component binding reagents are capable of binding to two different cellular components. Two of the plurality of cellular component binding reagents can be capable of binding to two different regions of a cellular component. The method can comprise, for each of the plurality of samples: (b) contacting the reagent oligonucleotides, associated with (or previously associated with) the cellular component binding reagents bound to the cellular components of (or from) the cells of the sample, with a plurality of beads (or particles) to obtaining reagent oligonucleotides bound to the plurality of beads. Each of the plurality of beads can comprise a bead dye. Each of the plurality of beads can comprise a plurality of bead oligonucleotides. Different beads of the plurality of beads can comprise different capture sequences for binding to different reagent-specific sequences of the reagent oligonucleotides. The method can comprise: (c) contacting the reagent oligonucleotides bound to the plurality of beads with a detection oligonucleotide to obtain reagent oligonucleotides bound to both the plurality of beads and the detection oligonucleotide. The detection oligonucleotide can comprise a detection dye. The detection oligonucleotide can comprise a binding sequence capable of binding to the detection sequences of the reagent oligonucleotides. The method can comprise: (d) determining an intensity of the bead dye and an intensity of the detection dye for each of the plurality of beads. The intensity of the bead dye and the intensity of the detection dye determined for a bead can indicate the identify and the quantity, respectively, of a cellular component of the cells of the sample. In some embodiments, the method comprises: determining the identity and the quantity of each of the cellular components based on the intensity of the bead dye and the intensity of detection dye, respectively, determined for each of the plurality of beads.

In some embodiments, providing the cells comprises: contacting the cells of each of the plurality of samples with the plurality of cellular component binding reagents to obtain the cells with the cellular components bound to the cellular component binding reagents. In some embodiments, providing the cells comprises: removing cellular component binding reagents of the plurality of cellular component binding reagents not bound to the cells to obtain the cells with the cellular components bound to the cellular component binding reagents.

There are provided, in some embodiments, methods of cellular component quantification. In some embodiments, the method comprises: contacting one or more cells of each of a plurality of samples with a plurality of cellular component binding reagents each associated with a reagent oligonucleotide, wherein two of the plurality of cellular component binding reagents are capable of binding to two different cellular components, or two different regions of a cellular component, and wherein each reagent oligonucleotide comprises (i) a reagent-specific sequence specific to the cellular component binding reagent associated thereto and (ii) a detection sequence, to obtain cells comprising cellular components bound to cellular component binding reagents of the plurality of cellular component binding reagents. The method can comprise: removing cellular component binding reagents of the plurality of cellular component binding reagents not bound to the cells. The method can comprise: contacting reagent oligonucleotides associated with cellular component binding reagents of the plurality of cellular component binding reagents not removed with a plurality of solid supports, wherein each of the plurality of solid supports is associated with one or more first detectable moieties, or precursors thereof, and comprises a plurality of solid support oligonucleotides, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences, to obtain reagent oligonucleotides bound to the plurality of solid supports. The method can comprise: contacting the reagent oligonucleotides bound to the plurality of solid supports with a detection oligonucleotide associated with one or more second detectable moieties, or precursors thereof and comprising a binding sequence capable of binding to the detection sequences of the reagent oligonucleotides to obtain reagent oligonucleotides bound to the plurality of solid supports and the detection oligonucleotide. The method can comprise: detecting the one or more first detectable moieties and the one or more second detectable moieties for each of the plurality of solid supports to determine the identity and the quantity, respectively, of each of the cellular components for each of the plurality of samples.

There are provided, in some embodiments, methods of cellular component quantification. In some embodiments, the method comprises: (a) providing one or more cells from each of a plurality of samples and with cellular components bound to cellular component binding reagents of a plurality of cellular component binding reagents (1) capable of binding to different cellular components, or regions thereof, and (2) each associated with a reagent oligonucleotide comprising (i) a reagent-specific sequence specific to the cellular component binding reagent associated thereto, and (ii) a detection sequence; and The method can comprise for each of the plurality of samples: (b) contacting reagent oligonucleotides, associated with or previously associated with the cellular component binding reagents bound to the cellular components of or from the cells of the sample, with a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of the reagent oligonucleotides, thereby obtaining reagent oligonucleotides bound to the plurality of solid supports; (c) contacting the reagent oligonucleotides bound to the plurality of solid supports with a detection oligonucleotide associated with one or more second detectable moieties, or precursors thereof, and comprising a binding sequence capable of binding to the detection sequences of the reagent oligonucleotides, thereby obtaining reagent oligonucleotides bound to both the plurality of solid supports and the detection oligonucleotide; and (d) detecting the one or more first detectable moieties and the one or more second detectable moieties for each of the plurality of solid supports to determine the identity and the quantity, respectively, of each of the cellular components for each of the plurality of samples.

Determining the identity and the quantity of each of the cellular components for each of the plurality of samples can comprises detecting the presence and/or amount of the one or more first detectable moieties and the one or more second detectable moieties for each of the plurality of solid supports. In some embodiments, the presence and/or amount of the one or more first detectable moieties and the presence and/or amount of the one or more second detectable moieties determined for a solid support indicate the identity and the quantity, respectively, of each of the cellular components for each of the plurality of samples. Detecting the presence and/or amount of the one or more first detectable moieties and the one or more second detectable moieties can comprise measuring emissions of the one or more first detectable moieties and the one or more second detectable moieties with an instrument, optionally measuring emissions using flow cytometry (e.g., fluorescence-activated cell sorting (FACS)). The method can comprise: (i) contacting two or more solid supports with two or more predetermined concentrations of a cellular component binding reagent, wherein each of the two or more solid supports is contacted with a different predetermined concentration of the cellular component binding reagent; (ii) contacting the two or more solid supports with the reagent oligonucleotides; and (iii) measuring emissions of the one or more second detectable moieties of each of the two or more first solid supports with an instrument to generate a calibration curve relating the quantity of at least one cellular component to emissions of the one or more second detectable moieties. The instrument can comprise a flow cytometer. The flow cytometer can comprise a conventional flow cytometer, a spectral flow cytometer, a hyperspectral flow cytometer, an imaging flow cytometer, or any combination thereof.

Contacting one or more cells of each of a plurality of samples with a plurality of cellular component binding reagents can comprise: partitioning the plurality of samples to a plurality of partitions, wherein a partition of the plurality of partitions comprises a single sample of the plurality of samples; and contacting one or more cells of each of a plurality of samples with a plurality of cellular component binding reagents. Contacting one or more cells of each of a plurality of samples with a plurality of cellular component binding reagents can comprise: contacting one or more cells of each of a plurality of samples with a plurality of cellular component binding reagents; and partitioning the plurality of samples to a plurality of partitions, wherein a partition of the plurality of partitions comprises a single sample of the plurality of samples.

Providing one or more cells from each of a plurality of samples and with cellular components bound to cellular component binding reagents of a plurality of cellular component binding reagents can comprise: providing a plurality of partitions each comprising a sample of the plurality of samples, wherein a partition of the plurality of partitions comprises a single sample of the plurality of samples. The plurality of samples can be partitioned to the plurality of partitions prior to contacting the one or more cells of each of a plurality of samples with a plurality of cellular component binding reagents, wherein a partition of the plurality of partitions comprises a single sample of the plurality of samples. The partition can be a well or a droplet. The plurality of partitions can comprise wells of a well array. The well array can comprise at least about 10 to 100 wells. The instrument can comprise a fluorescence microscope. The instrument can comprise an imaging system. Measuring emissions of each detectable moiety of each first solid support can comprise imaging the plurality of partitions. The plurality of partitions can be imaged sequentially. The plurality of partitions can be imaged simultaneously. Imaging can comprise microscopy, confocal microscopy, time-lapse imaging microscopy, fluorescence microscopy, multi-photon microscopy, quantitative phase microscopy, surface enhanced Raman spectroscopy, videography, manual visual analysis, automated visual analysis, or any combination thereof.

In some embodiments, detecting the one or more first detectable moieties and the one or more second detectable moieties for each of the plurality of solid supports comprises: detecting the one or more first detectable moieties and the one or more second detectable moieties for each of the plurality of solid supports of each sample of the plurality of samples separately, thereby determining the identity and the quantity, respectively, of each of the cellular components for each sample of the plurality of samples. In some embodiments, detecting the one or more first detectable moieties and the one or more second detectable moieties for each of the plurality of solid supports of each sample of the plurality of samples separately comprises detecting the one or more first detectable moieties and the one or more second detectable moieties for each of the plurality of solid supports of each partition separately.

The one or more first detectable moieties of the plurality of solid supports situated in each partition can be predetermined, and said predetermined one or more first detectable moieties can be distinct to each partition. In some embodiments, detecting the one or more first detectable moieties and the one or more second detectable moieties for each of the plurality of solid supports comprises: detecting the predetermined one or more first detectable moieties and the one or more second detectable moieties for each of the plurality of solid supports of each sample of the plurality of samples concurrently; and associating the detected predetermined one or more first detectable moieties of each of the solid supports with the partition from which said solid support derived, thereby determining the identity and the quantity, respectively, of each of the cellular components for each sample of the plurality of samples. The method can comprise: pooling the solid supports from each partition of the plurality of partitions (e.g., using a magnetic field). In some embodiments, contacting the reagent oligonucleotides bound to the plurality of solid supports with the detection oligonucleotide comprises: contacting the reagent oligonucleotides bound to the plurality of solid supports with two or more detection oligonucleotides each associated with one or more second detectable moieties.

The two or more detection oligonucleotides can be associated with an identical second detectable moieties. The two or more detection oligonucleotides can be associated with different second detectable moieties. The two or more detection oligonucleotides can comprise an identical binding sequence. The two or more detection oligonucleotides can comprise different binding sequences. In some embodiments, each of the plurality of solid supports is associated with two distinct first detectable moieties. Two solid supports of the plurality of solid supports can comprise different types and/or quantities of the two distinct first detectable moieties. The method can comprise: isolating one or more populations of interest from a starting population to obtain the plurality of samples. Each of the samples can be a population of interest. Two or more of the samples of the plurality of samples can comprise phenotypically different populations of interest. Isolating one or more populations of interest from a starting population can comprise flow cytometry (e.g., fluorescence-activated cell sorting (FACS)). The method can comprise: isolating the one or more cells from the sample. Isolating the one or more cells can comprise isolating the one or more cells from the sample using flow cytometry (e.g., fluorescence-activated cell sorting (FACS)). The method can comprise: lysing the cells, prior to contacting the reagent oligonucleotides with the plurality of solid supports. The method can comprise: dissociating the reagent oligonucleotides from the cellular component binding reagents bound to or previously bound to the cellular components of or from the cells of the sample, prior to contacting the reagent oligonucleotides with the plurality of solid supports.

At least two solid support oligonucleotides of a solid support of the plurality of solid supports can comprise an identical capture sequence for binding to one of the reagent-specific sequences. A solid support oligonucleotide of a first solid support and a solid support oligonucleotide of a second solid support of the plurality of solid supports can comprise different capture sequences for binding to two different reagent-specific sequences of the reagent-specific sequences. Two solid supports of the plurality of solid supports can comprise different quantities of the one or more first detectable moieties. Two solid supports of the plurality of solid supports can comprise different first detectable moieties. All solid supports of the plurality of solid supports can be distinguishable from each other by the presence and/or amount of the one or more first detectable moieties associated thereto. The one or more first detectable moieties can be attached, releasably attached, covalently attached, non-covalently attached, and/or conjugated to the solid support.

### Cellular Component Binding Reagents Binding

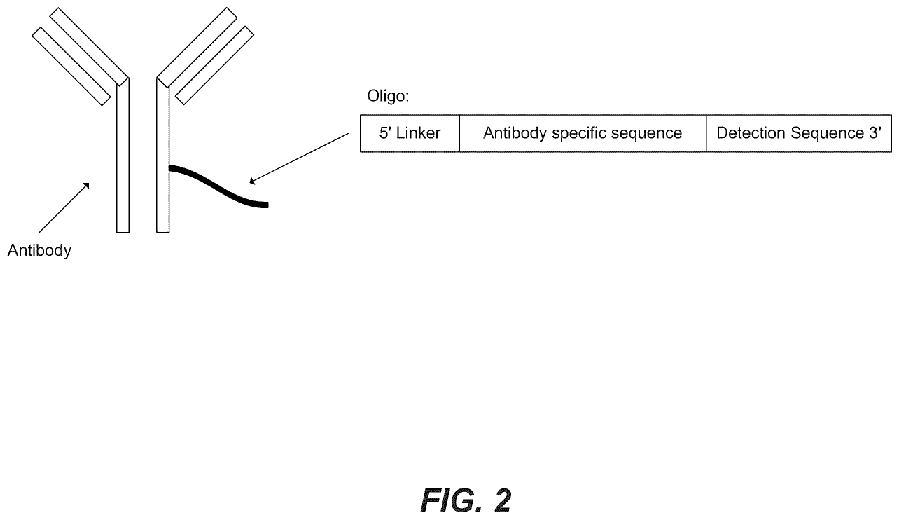

In some embodiments, a method of cellular component quantification comprises: contacting one cell, one or more cells, or each cell of one sample, one or more samples, or each sample of a plurality of samples with a plurality of cellular component binding reagents (or different cellular component binding reagents or molecules of each of a plurality of cellular component binding reagents) to obtain cells comprising cellular components (e.g., proteins) bound to cellular component binding reagents (e.g., antibodies) of the plurality of cellular component binding reagents. FIG. 2 illustrates a cellular component binding reagent that is an antibody. Cells comprising cellular components bound to cellular component binding reagents can be referred to herein as stained cells. The number of cells in a sample can be different in different embodiments. In some embodiments, the number of cells in a sample is, is about, is at least, is at least about, is at most, or is at most about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values. The number of different samples can be different in different embodiments. In some embodiments, the number of different samples is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values. Cellular component binding reagents (such as barcoded antibodies) and their uses (such as sample indexing of cells) have been described in U.S. Patent Application Publication Nos. US2018/0088112 and US2018/0346970; the content of each of these is incorporated herein by reference in its entirety.

In some embodiments, the plurality of cellular component binding reagents comprises a protein, an antibody, an aptamer, a tetramer, a protein scaffold, or a combination thereof. The aptamer and the reagent oligonucleotide can be a single polynucleotide. The aptamer can be, 10, 20, 30, 40, 50, 60, 70, 80, 90, 100, 200, 300, 400, 500, 600, 700, 800, 900, 1000, or a number or a range between any two of these values, nucleotides in length. The number of different cellular component binding reagents can be different in different embodiments. In some embodiments, the number of different cellular components binding reagents is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values. One, one or more, or each of the plurality of cellular component binding reagents can be associated with (e.g., attached with or conjugated with) a reagent oligonucleotide (also referred to herein as a barcode oligonucleotide). The number of cellular component binding reagents each associated with a reagent oligonucleotide can be different in different embodiments. In some embodiments, the number of cellular components binding reagents each associated with a reagent oligonucleotide is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values.

Two (or two or more) of the plurality of cellular component binding reagents are capable of binding to two different cellular components, or two different regions (e.g., epitopes) of a cellular component. The number of different cellular components the plurality of cellular component binding reagents is capable of binding to can be different in different embodiments. In some embodiments, the number of different cellular components the plurality of cellular component binding reagent is capable of binding is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values. Alternatively or additionally, each of the plurality of cellular component binding reagents are capable of binding to a different cellular components.

In some embodiments, the cellular components comprise a protein, a lipid, a carbohydrate, or a combination thereof. The cellular components can comprise an extracellular cellular component, a cell surface cellular component, an intracellular cellular component, or a combination thereof. The number of different cellular components can be different in different embodiments. In some embodiments, the number of different cellular components is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values.

The length of the reagent oligonucleotide can vary. In some embodiments, the reagent oligonucleotide is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the reagent oligonucleotide can be 10 to 500 nucleotides in length.

Each reagent oligonucleotide can comprise a reagent-specific sequence specific to the cellular component binding reagent associated with the reagent oligonucleotide. The length of the reagent-specific sequence can be different in different embodiments. In some embodiments, the reagent-specific sequence is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the reagent-specific sequence is 5 to 495 nucleotides in length. The number of different reagent-specific sequences specific to the cellular component binding reagents can be different in different embodiments. In some embodiments, the number of different reagent-specific sequences specific to the cellular component binding reagents is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 2000, 3000, 4000, 5000, 6000, 7000, 8000, 9000, 10000, or a number or a range between any two of these values.

Each reagent oligonucleotide can comprise a detection sequence. The length of the detection sequence can be different in different embodiments. In some embodiments, the detection sequence is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the detection sequence is 5 to 495 nucleotides in length.

In some embodiments, a cellular component binding reagent is associated with (e.g., attached with or conjugated with) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, reagent oligonucleotides. The reagent oligonucleotides of a cellular component binding reagent can comprise an identical sequence. The reagent oligonucleotides of a cellular component binding reagent can comprise different sequences, such as different reagent-specific sequences and/or different detection sequences.

In some embodiments, the reagent oligonucleotide is attached, releasably attached, covalently attached, non-covalently attached, and/or conjugated to the cellular component binding reagent. In some embodiments, the reagent oligonucleotide is associated with the cellular component through a cleavable or labile group, such as a UV photocleavable group, a chemical labile group (e.g., a disulfide bond), or a heat labile group. The reagent oligonucleotide can dissociate (e.g., detach) from the cellular component by a chemical stimulus, a physical stimulus, a biological stimulus, a thermal stimulus, a magnetic stimulus, an electric stimulus, a light stimulus, or any combination thereof.

In some embodiments, the reagent oligonucleotide is associated with the cellular component through a linker. The linker can comprise a carbon chain. The linker or the carbon chain can comprise 2-30 carbons, such as 12 carbons. In some embodiments, the linker or the carbon chain comprises, comprises about, comprises at least, comprises at least about, comprises at most, or comprises at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, or a number or a range between any two of these values, carbons. The linker can comprise 5′ amino modifier C12 (5AmMC12), or a derivative thereof.

A reagent oligonucleotide can comprise different numbers of reagent-specific sequences in different embodiments. In some embodiments, a reagent oligonucleotide comprises, comprises about, comprises at least, comprises at least, comprises at most, or comprises at most, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, reagent-specific sequences. A reagent oligonucleotide can comprise different numbers of detection sequences in different embodiments. For example, a reagent oligonucleotide comprises two or more reagent-specific sequences. Two (or two or more, such as 3, 4, 5, 6, 7, 8, 9, 10, 20, 30, 40, 50, 60, 70, 80, 90, 100) reagent-specific sequences of a reagent oligonucleotide can comprise the same sequence. Two (or two or more, such as 3, 4, 5, 6, 7, 8, 9, 10, 20, 30, 40, 50, 60, 70, 80, 90, 100) reagent-specific sequences of a reagent oligonucleotide can comprise different sequences. In some embodiments, a reagent oligonucleotide comprises, comprises about, comprises at least, comprises at least, comprises at most, or comprises at most, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, detection sequences. For example, a reagent oligonucleotide comprises two or more detection sequences. Two (or two or more, such as 3, 4, 5, 6, 7, 8, 9, 10, 20, 30, 40, 50, 60, 70, 80, 90, 100) detection sequences of a reagent oligonucleotide can comprise the same sequence. Two (or two or more, such as 3, 4, 5, 6, 7, 8, 9, 10, 20, 30, 40, 50, 60, 70, 80, 90, 100) detection sequences of a reagent oligonucleotide can comprise different sequences. In some embodiments, a reagent oligonucleotide has a hairpin structure.

The method can comprise: removing cellular component binding reagents of the plurality of cellular component binding reagents not bound to the cells. Removing the cellular component binding reagents not bound to the cells can comprise washing the cells with a washing buffer.

In some embodiments, the method comprises: isolating the one or more cells from the sample prior to contacting the cells of each of the plurality of samples with the plurality of cellular component binding reagents. In some embodiments, the method comprises: isolating the one or more cells from the sample subsequent to contacting the cells of each of the plurality of samples with the plurality of cellular component binding reagents. Isolating the one or more cells can comprise: isolating the one or more cells from the sample using flow cytometry, such as fluorescence-activated cell sorting (FACS).

### Fixating and Permeabilizing

In some embodiments, the method comprises: fixating (e.g., PFA, formalin) and membrane permeabilization (e.g., saponin permeabilization) of the cells prior to contacting the cells of each of the plurality of samples with the plurality of cellular component binding reagents. Fixation can be performed using a fixating agent, such as a cross-linking agent. The fixing agent can comprise a cleavable cross-linking agent. The cleavable cross-linking agent can comprise a thiol-cleavable cross-linking agent. The cleavable cross-linking agent can comprise or be derived from dithiobis(succinimidyl propionate) (DSP, Lomant's Reagent), disuccinimidyl tartrate (DST), Bis [2-(Succinimidooxycarbonyloxy)ethyl] Sulfone (BSOCOES), ethylene glycol bis(succinimidyl succinate) (EGS), dimethyl 3,3′-dithiobispropionimidate (DTBP, Wang and Richard's Reagent), succinimidyl 3-(2-pyridyldithio)propionate (SPDP), succinimidyl 6-(3(2-pyridyldithio)propionamido)hexanoate (LC-SPDP), 4-succinimidyloxycarbonyl-alpha-methyl-α(2-pyridyldithio)toluene (SMPT), 3-(2-pyridyldithio)propionyl hydrazide (PDPH), succinimidyl 2-((4,4′-azipentanamido)ethyl)-1,3′-dithiopropionate (SDAD, NHS—SS-Diazirine), or any combination thereof. The cleavable cross-linking agent can comprise a cleavable linkage selected from a chemically cleavable linkage, a photocleavable linkage, an acid labile linker, a heat sensitive linkage, an enzymatically cleavable linkage, or any combination thereof. The cleavable cross-linking agent can comprise a disulfide linker. The fixing agent can comprise BD Cytofix or a reversible cross-linker. The fixing agent can comprise a non-cross-linking fixative, optionally the non-cross-linking fixative comprises methanol. The method can comprise contacting the cells with an unfixing agent. The unfixing agent can comprise a thiol, hydoxylamine, periodate, a base, or any combination thereof. The unfixating agent can comprise DTT. The unfixing agent can, in some embodiments, cleave a disulfide bridge. The unfixing agent can reverse the fixation during a lysis step.

Membrane permeabilization can be reversible or irreversible. Fixation can be reversible or irreversible. The permeabilizing agent can be capable of (i) permeabilizing the cell membranes of the cells; and/or (ii) making a cell membrane permeable to the cellular component binding reagents. The permeabilizing agent can comprise (i) a solvent, a detergent, or a surfactant; (ii) BD Cytoperm; (iii) a saponin or a derivative thereof; and/or (iv) digitonin or a derivative thereof. The method can comprise removing the permeabilizing agent (e.g, removing saponin). Removal of the permeabilizing agent can refill the membrane (e.g., reconstitute membrane integrity).

### Oligonucleotide Capture

The method can comprise: contacting the reagent oligonucleotides associated with (or previously associated with) cellular component binding reagents of the plurality of cellular component binding reagents not removed (or cellular component binding reagents of the plurality of cellular component binding reagents bound to the cellular components of (or from) the cells of the sample) with a plurality of solid supports (or a plurality of different solid supports, such as molecules of each of a plurality of solid supports), such as particles to obtain reagent oligonucleotides bound to the plurality of solid supports. One, one or more, or each of the plurality of solid supports can comprise (e.g., associated with, such as attached with or coupled with) one or more first detectable moieties (also referred to herein as a bead dye, or a clustering dye), such as a fluorescent dye. Each of the plurality of solid supports can comprise (e.g., attached to) a plurality of solid support oligonucleotides (also referred to herein as clustering oligonucleotides). Solid support oligonucleotides associated with a solid support can be identical or different.

The plurality of solid supports can comprise different numbers of solid supports in different implementations. In some embodiments, the plurality of solid supports comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, solid supports. For example, the plurality of solid supports comprises at least 10 solid supports.

One, one or more, or each of the plurality of solid supports can comprise (e.g., associated with, such as attached with or coupled with) one or more first detectable moieties (also referred to herein as bead dye or clustering dye), such as a fluorescent dye. In some embodiments, all solid supports of the plurality of solid supports are distinguishable from each other by intensities of the bead dye associated with the plurality of beads. In some embodiments, the bead dye is attached, releasably attached, covalently attached, non-covalently attached, and/or conjugated to the bead. Solid supports comprising solid support oligonucleotides with different capture sequences can have different quantities of a bead dye (thus with different quantities of fluorescence, for example). The number of solid supports with different quantities of a bead dye can be different in different embodiments. In some embodiments, the number of solid supports with different quantities of a bead dye is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. For example, two solid supports of the plurality of solid supports comprise different quantities of a bead dye. The number of solid supports with different bead dyes can be different in different embodiments. In some embodiments, the number of solid supports with bead dyes is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. For example, two solid supports of the plurality of solid supports comprise different bead dyes.

In some embodiments, a solid support is associated with two or more bead dyes, such as 2, 3, 4, 5, 6, 7, 8, 9, 10, bead dyes. The number of solid supports with the same quantity of a first bead dye of the two bead dyes can be different in different embodiments. In some embodiments, the number of solid supports with the same quantity of a first bead dye (or a second bead dye) of the two bead dyes is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. The number of solid supports with the different quantities of a first bead dye of the two bead dyes can be different in different embodiments. In some embodiments, the number of solid supports with different quantities of a first bead dye (or a second bead dye) of the two bead dyes is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. For example, two solid supports of the plurality of solid supports can comprise different quantities of a first bead dye of the two bead dyes. For example, two solid supports of the plurality of solid supports can comprise different quantities of a second bead dye of the two bead dyes. The number of solid supports with different combinations of quantities of the two bead dyes can be different in different embodiments. In some embodiments, the number of solid supports with different combinations of quantities of the two bead dyes is, is about, is at least, is at least about, is at most, or is at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values. All solid supports of the plurality of solid supports can be distinguishable from each other by the combinations of intensities (e.g., fluorescent intensities) of the bead dyes associated with the plurality of solid supports.

Each of the plurality of solid supports can comprise (e.g., attached to) a plurality of solid support oligonucleotides (also referred to herein as clustering oligonucleotides). The number of solid support oligonucleotides attached to a solid support can be different in different embodiments, In some embodiments, the number of solid support oligonucleotides attached to a solid support is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, or a number or a range between any two of these values.

The length of the solid support oligonucleotide can vary. In some embodiments, the solid support oligonucleotide is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the solid support oligonucleotide is 10 to 500 nucleotides in length.

A solid support oligonucleotide can comprise a capture sequence. The length of the capture sequence can be different in different embodiments. In some embodiments, the capture sequence is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the capture sequence is 5 to 490 nucleotides in length.

At least two solid support oligonucleotides of a solid support of the plurality of solid supports can comprise an identical capture sequence for binding to one of the reagent-specific sequences. The number of solid support oligonucleotides of a solid support comprising an identical capture sequence can be different in different embodiments. In some embodiments, the number of solid support oligonucleotides of a solid support comprising an identical capture sequence is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, or a number or a range between any two of these values.

A solid support oligonucleotide of a first solid support and a solid support oligonucleotide of a second solid support of the plurality of solid supports can comprise different capture sequences for binding to two different reagent-specific sequences of the reagent-specific sequences. The number of solid supports with solid support oligonucleotides with different capture sequences can be different in different embodiments. In some embodiments, the number of solid supports with solid support oligonucleotides with different capture sequences is, is about, is at least, is at least about, is at most, or is at most about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, or a number or a range between any two of these values.

### Lysis

In some embodiments, the method comprises: lysing the cells, prior to contacting the reagent oligonucleotides with the plurality of solid supports. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, 10, or more. The pH of the lysis buffer can be at most about 1, 2, 3, 4, 5, 6, 7, 8, 9, 10, or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7%, or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT.

Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

### Oligonucleotide Dissociation

In some embodiments, the method comprises: dissociating the reagent oligonucleotides from the cellular component binding reagents bound to (or previously bound to) the cellular components of (or from) the cells of the sample, prior to contacting the reagent oligonucleotides with the plurality of solid supports. Dissociating the reagent oligonucleotides can comprise: detaching the reagent oligonucleotides from the cellular component binding reagents bound to (or previously bound to) the cellular components of (or from) the cells of the sample using UV photocleaving, chemical treatment, heat treatment, enzyme treatment, or a combination thereof. Dissociating the reagent oligonucleotides can comprise: detaching the reagent oligonucleotides from the cellular component binding reagents bound to (or previously bound to) the cellular components of (or from) the cells of the sample using a chemical stimulus, a physical stimulus, a biological stimulus, a thermal stimulus, a magnetic stimulus, an electric stimulus, a light stimulus, or any combination thereof.

### Detection

Determining the identity and the quantity of each of the cellular components for each of the plurality of samples can comprises detecting the presence and/or amount of the one or more first detectable moieties (e.g., bead dye(s)) and the one or more second detectable moieties (e.g., detection dye(s)) for each of the plurality of solid supports (e.g., beads). The method can comprise: contacting the reagent oligonucleotides bound to the plurality of beads with a detection oligonucleotide (also referred to herein as a reporter oligonucleotide), such as molecules of a detection oligonucleotide, to obtain reagent oligonucleotides bound to both the plurality of beads and the detection oligonucleotide (a sandwich of reagent oligonucleotides bound to both the plurality of beads and the detection oligonucleotide). The detection oligonucleotide can comprise (e.g., associated with, such as attached with or coupled with) a detection dye (also referred to herein as a reporter oligonucleotide), such as a fluorescent dye. In some embodiments, the detection dye is attached, releasably attached, covalently attached, non-covalently attached, and/or conjugated to the detection oligonucleotide.

The detection oligonucleotide can comprise a binding sequence capable of binding to the detection sequences of the reagent oligonucleotides. The length of the detection oligonucleotide can be different in different embodiments. In some embodiments, the detection oligonucleotide is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the detection oligonucleotide is 10 to 500 nucleotides in length. The length of the binding sequence can be different in different embodiments. In some embodiments, the binding sequence is about, is at least, is at least about, is at most, or is at most about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, nucleotides in length. For example, the binding sequence is 5 to 490 nucleotides in length.

In some embodiments, contacting the reagent oligonucleotides bound to the plurality of beads with the detection oligonucleotide comprises: contacting the reagent oligonucleotides bound to the plurality of beads with two or more (such as 2, 3, 4, 5, 6, 7, 8, 9, 10, 20, 30, 40, 50, 60, 70, 80, 90, or 100) detection oligonucleotides each associated with a detection dye. Two or more (such as 2, 3, 4, 5, 6, 7, 8, 9, 10, 20, 30, 40, 50, 60, 70, 80, 90, or 100) detection oligonucleotides can be associated with an identical detection dye. Two or more (such as 2, 3, 4, 5, 6, 7, 8, 9, 10, 20, 30, 40, 50, 60, 70, 80, 90, or 100) detection oligonucleotides can be associated with different detection dyes. Two or more (such as 2, 3, 4, 5, 6, 7, 8, 9, 10, 20, 30, 40, 50, 60, 70, 80, 90, or 100) detection oligonucleotides can comprise an identical binding sequence. Two (such as 2, 3, 4, 5, 6, 7, 8, 9, 10, 20, 30, 40, 50, 60, 70, 80, 90, or 100) or more detection oligonucleotides can comprise different binding sequences.

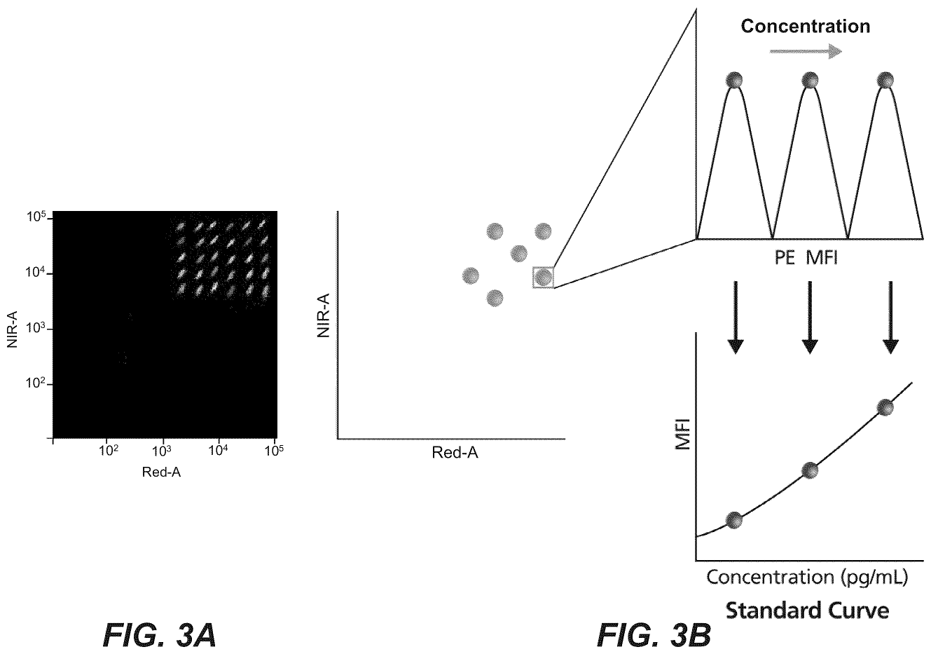

An array, such as the virtual multidimensional array illustrated in FIGS 3A-3B, can contain a plurality of bead populations, wherein different populations are labeled, using the same two fluorophores, at a plurality of discrete fluorescence levels for each of the two fluorophores. The fluorescence properties of the arrays can enable the identification of the beads in each population by exposing the array to excitation light and measuring the fluorescence of each bead in each of two detection channels, one for each of the two fluorophores. The beads in the array can be detected and uniquely identified by exposing the beads to excitation light and measuring the fluorescence of each bead in each of the two detection channels. The excitation light can be from one or more light sources and can be either narrow or broadband. Examples of excitation light sources include lasers, and light emitting diodes. For identification of beads in the arrays, two detector channels can be used. The detector channels can be non-overlapping channels or partially overlapping. A flow cytometer can have two channels used to detect bead fluorescence and a third channel is used to detect reporter fluorescence.

The beads from the reaction mixture can be flow cytometrically assayed to detect the presence and/or to determine the quantity of the bead dye. Flow cytometry uses multi-parameter data for identifying and distinguishing between different particle types (i.e., particles that vary from one another terms of label (wavelength, intensity), size, etc., in a fluid medium. A liquid medium comprising the beads can first be introduced into the flow path of the flow cytometer. When in the flow path, the beads can pass substantially one at a time through one or more sensing regions, where each of the beads is exposed individually to a source of light at a single wavelength (or a single source of light at multiple wavelengths, or multiple sources of light at multiple wavelengths) and measurements of light scatter parameters and/or fluorescent emissions as desired (e.g., two or more light scatter parameters and measurements of one or more fluorescent emissions) are separately recorded for each bead. The data recorded for each bead can be analyzed in real time or stored in a data storage and analysis means, such as a computer, as desired.

Detectors (e.g., light collectors, such as photomultiplier tubes (or “PMT”)) in a sensing region can record light that passes through each bead (generally referred to as forward light scatter), light that is reflected orthogonal to the direction of the flow of the beads through the sensing region (referred to as orthogonal or side light scatter) and fluorescent light emitted from the beads, if the bead is labeled with fluorescent marker(s), as the bead passes through the sensing region and is illuminated by the energy source. Each of forward light scatter (or FSC), orthogonal light scatter (SSC), and fluorescence emissions (FL1, FL2, etc.) can comprise a separate parameter for each bead (or each “event”). Thus, for example, two, three or four parameters can be collected (and recorded) by two, three or four detectors from a bead labeled with two different fluorescence markers. In some embodiments, beads from two bead populations can have different sizes, such that the beads can be distinguished by their FSC and SSC characteristics.

A particle array can include populations of microparticles (e.g., beads), wherein each microparticle is labeled with a single fluorescent dye. The array can comprise a plurality of particle populations. In some embodiments, particle populations are labeled, using the same fluorophore, such that each population exhibits a measurably distinct mean fluorescence intensity. In some embodiments, particle populations in different sets of particle populations are labeled with different fluorescent dyes, wherein all of the fluorescent dyes can be excited by the same excitation light, the emission spectra of each dye is detectable using the same two detection channels, and the relative amount of emissions in each of the two detection channels is distinguishably distinct between different dyes.

Fluorescence emitted in detection channels used to identify the particles can be measured following excitation with a single light source, or can be measured separately following excitation with distinct light sources. If separate excitation light sources are used to excite the particle dyes, the dyes preferably are selected such that all the dyes used to construct the array are excitable by each of the excitation light sources used. For example, a dual laser flow cytometer can have 488 nm and 635 nm excitation lasers that are focused on the flow stream at spatially discrete regions, and detection optics designed to measure light in three detection channels, designated FL1, FL2, and FL3, following excitation by the 488 nm laser, and a fourth detection channel, designated FL4, following excitation by the 635 nm laser. In some embodiments, FL3 and FL4 are selected as the two detection channels used to identify the particle populations. For example, one channel, FL3, can be measured following excitation by the 488 nm laser and the second channel, FL4, can be measured following excitation by the 635 nm laser. The selection of dyes and detection channels in can be made in view of the configuration of an existing commercial instrument. Alternatively, a flow cytometer could be configured to measure emission in both FL3 and FL4 following excitation with a single laser.

### Analysis

Detecting the presence and/or amount of the one or more first detectable moieties and the one or more second detectable moieties can comprise measuring emissions of the one or more first detectable moieties (e.g., bead dye(s)) and the one or more second detectable moieties (e.g., detection dye(s)) with an instrument. The method can comprise: determining an intensity of the bead dye (e.g., fluorescent intensity) and an intensity (e.g., fluorescent intensity) of the detection dye for each of the plurality of beads. The plot in FIG. 1 shows six populations of intensities of a single bead dye corresponding to six beads (or six different types of beads), labeled a1 to a6. FIG. 1 shows the quantity of the detection dye is about 10³ for bead a1 and between 10⁰ to 10¹ for beads a2 to a6. The method can comprise: determining a quantity of each of the cellular components based on the intensity of the bead dye (to determine the identity of each cellular component) and the intensity of the detection dye (to determine the quantity of the cellular component) determined for each of the plurality of beads. The quantity of a cellular component can be determined using, for example, a standard curve (see FIG. 3B right hand side for an example) of the intensity of the detection dye and the quantity (e.g., the concentration) of the detection oligonucleotide which corresponds to the corresponding cellular component. The intensity of the detection dye depends on the quantity (or the number of molecules) of the bead dye and can be used to determine (or correlates with) the identity of the bead; the capture sequences (which can be identical) of the bead oligonucleotides of the bead; the reagent-specific sequences (which can be identical) of the reagent oligonucleotides that bind to, or captured by, the bead oligonucleotides of the bead (or molecules of the reagent-specific sequence of the reagent oligonucleotide that bind to, or captured by, the bead oligonucleotides of the bead); the cellular component binding reagents associated with or previously associated with the bead oligonucleotides (or molecules of the cellular component binding reagent associated with or previously associated with molecules of the bead oligonucleotide); and the identity of the cellular component bound to or previously bound to the cellular component binding reagent. The intensity of the detection dye depends on the number of molecules of the detection dye and the number of molecules of the detection oligonucleotide comprising the bead dye. The intensity of the detection dye can be used to determine (or correlates with) the quantity (or the number of molecules) of cellular component binding reagent with the reagent-specific oligonucleotide captured by/bound to both bead oligonucleotides of the bead and the detection oligonucleotide comprising the detection dye.

In some embodiments, determining the intensity of the bead dye and the intensity of the detection dye comprises: determining the intensity of the bead dye and the intensity of the detection dye for each of the plurality using flow cytometry, such as fluorescence-activated cell sorting (FACS).

In some embodiments, determining the intensity of the bead dye can comprise: determining the intensities of the two (or more) bead dyes for each of the plurality of beads (FIGS. 3A-3B illustrates bead dyes NIR-A and Red-A). Determining the quantity of each of the cellular components comprises: determining the quantity of each of the cellular components based on the combination of the intensities (or a two-dimensional array of intensities) of the bead dyes (FIG. 3A shows 30 combinations of intensities of the two bead dyes; FIG. 3B left hand side shows six combinations of intensities of the two bead dyes) and the intensity of the detection dye determined for each of the plurality of beads.

The fluorescence intensity data with respect to the two fluorophores (and channels) can be plotted in a two-dimensional dot-plot, with intensity of the two detection channels on the two axes (See FIGS. 3A-3B for an illustration). Each population can appear as cluster uniquely positioned in the two-dimensional dot plot. The two fluorophores used for identifying the beads (and thus the analytes bound to the beads) are referred to herein as clustering fluorescent dyes. The fluorescent intensities of the two fluorophores detected by, for example, the two detection channels, are referred to herein as clustering fluorescence intensities. The identity of the particle populations, determined from the particle fluorescence measured in the two detection channels, enables identification of the analyte bound to the particle through the analyte-specific reagent.

The quantity of each reagent oligonucleotide (or the corresponding cellular component) can be determined based on the fluorescence intensities from molecules of a reporter reagent (e.g., a detection oligonucleotide comprising a detection dye) for the reagent oligonucleotide. For example, the median fluorescence intensity (MFI) of the fluorescence intensities from molecules of the reporter reagent for the analyte can be used to determine the concentration of the reagent oligonucleotide (or the corresponding cellular component) using a standard curve. A standard curve of the correspondence between the median fluorescence intensity (MFI) and the concentration of the reagent oligonucleotide (or the corresponding cellular component) can be determined as disclosed herein using samples of known concentrations of the reagent oligonucleotide (or the corresponding cellular component). The quantities of reagent oligonucleotides (or the corresponding cellular components) can be determined based on the fluorescence intensities of the single fluorescent dye conjugated to the reporter reagents. For example, the median fluorescence intensities (MFIs) of the fluorescence intensities from molecules of the reporter reagents for the reagent oligonucleotides can be used to determine the concentration of the reagent oligonucleotides (or the corresponding cellular component) using standard curves. Standard curves of the correspondence between the MFIs and concentration of reagent oligonucleotides (or the corresponding cellular components) can be determined using samples of known concentrations of the reagent oligonucleotides (or the corresponding cellular components).

### Amplification

The method can comprise: amplifying (e.g., using polymerase chain reaction) the reagent oligonucleotides (or molecules of the reagent oligonucleotides) associated with (or previously associated with) the cellular component binding reagents bound to the cellular components of (or from) the cells of the sample to obtain amplified reagent oligonucleotides. The amplification can include 1, 2, 3, 4, 5, 6, 7, 8, 9, 10, 11, 12, 13, 14, 15, 16, 17, 18, 19, 20, 25, 30, 35, 40, 45, 50, or a number or a range between any two of these values, cycles of amplification. Amplification of the labeled nucleic acids can comprise PCR-based methods or non-PCR based methods.

Contacting the reagent oligonucleotides, associated with (or previously associated with) the cellular component binding reagents bound to the cellular components of (or from) the cells of the sample, can comprise: contacting the amplified reagent oligonucleotides (or molecules of the amplified reagent oligonucleotides) with the plurality of solid supports (e.g., beads) to obtain amplified reagent oligonucleotides bound to the plurality of solid supports (e.g., beads). Contacting the reagent oligonucleotides bound to the plurality of solid supports (e.g., beads) can comprise: contacting the amplified reagent oligonucleotides bound to the plurality of solid supports (e.g., beads) with the detection oligonucleotide to obtain amplified reagent oligonucleotides bound to both the plurality of solid supports (e.g., beads) and the detection oligonucleotide (also referred to herein as a sandwich).

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Amplification can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the reagent oligonucleotides. The one or more primers can anneal to the 3′ end or 5′ end of the reagent oligonucleotides. The one or more primers can anneal to an internal region of the reagent oligonucleotides. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ (or 5′) ends the reagent oligonucleotides.

### Kits

Disclosed herein include kits for cellular component quantification. In some embodiments, a kit for cellular component quantification comprises: a plurality of cellular component binding reagents (or molecules of each of a plurality of cellular component binding reagents). The plurality of cellular component binding reagents is capable of binding to different cellular components, or regions thereof. Each of the plurality of cellular component binding reagents is associated with a reagent oligonucleotide comprising (i) a reagent-specific sequence specific to a cellular component binding reagent associated with the reagent oligonucleotide, and (ii) a detection sequence. The kit can comprise: a plurality of beads (or molecules of each of a plurality of beads). Each of the plurality of beads can be associated with (e.g., attached with) a bead dye and comprises a plurality of bead oligonucleotides. Different beads of the plurality of beads can comprise different capture sequences for binding to different reagent-specific sequences of reagent oligonucleotides. The kit can comprise: a detection oligonucleotide (or molecules of a detection oligonucleotide). The detection oligonucleotide can be associated with a detection dye. The detection oligonucleotide can comprise a binding sequence capable of binding to the detection sequences of the reagent oligonucleotides.

Disclosed herein include kits. In some embodiments, the kit comprises: a plurality of cellular component binding reagents (1) capable of binding to different cellular components, or regions thereof, and (2) each associated with a reagent oligonucleotide comprising (i) a reagent-specific sequence specific to a cellular component binding reagent associated with the reagent oligonucleotide, and (ii) a detection sequence. The kit can comprise: a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of reagent oligonucleotides. The method can comprise: a detection oligonucleotide associated with one or more second detectable moieties, or precursors thereof, and comprising a binding sequence capable of binding to the detection sequences of the reagent oligonucleotides.

### Antibody

An antibody can be humanized or chimeric. The antibody can be a naked antibody or a fusion antibody. The antibody can be a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. The antibody fragment can be, for example, a portion of an antibody such as F(ab′)2, Fab′, Fab, Fv, sFv and the like. In some embodiments, the antibody fragment can bind with the same antigen that is recognized by the full-length antibody. The antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (CD8, CD34, CD45), and therapeutic antibodies.

### Particle

A bead (or a particle) can be a solid support. The bead can be, for example, a silica gel bead, a controlled pore glass bead, a magnetic bead, a Dynabead, a Sephadex/Sepharose bead, a cellulose bead, a polystyrene bead, or any combination thereof. The bead can comprise a material such as polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, or any combination thereof.

In some embodiments, the bead can be a polymeric bead, for example a deformable bead or a gel bead, functionalized with barcodes or stochastic barcodes. In some implementation, a gel bead can comprise a polymer based gels. Gel beads can be generated, for example, by encapsulating one or more polymeric precursors into droplets. Upon exposure of the polymeric precursors to an accelerator (e.g., tetramethylethylenediamine (TEMED)), a gel bead may be generated.

In some embodiments, the particle can be degradable. For example, the polymeric bead can dissolve, melt, or degrade, for example, under a desired condition. The desired condition can include an environmental condition. The desired condition may result in the polymeric bead dissolving, melting, or degrading in a controlled manner. A gel bead may dissolve, melt, or degrade due to a chemical stimulus, a physical stimulus, a biological stimulus, a thermal stimulus, a magnetic stimulus, an electric stimulus, a light stimulus, or any combination thereof.

Bead oligonucleotides, for example, may be coupled/immobilized to the interior surface of a gel bead (e.g., the interior accessible via diffusion of an oligonucleotide barcode and/or materials used to generate an oligonucleotide barcode) and/or the outer surface of a gel bead or any other microcapsule described herein. Coupling/immobilization may be via any form of chemical bonding (e.g., covalent bond, ionic bond) or physical phenomena (e.g., Van der Waals forces, dipole-dipole interactions, etc.). In some embodiments, coupling/immobilization of bead oligonucleotides to a gel bead or any other microcapsule described herein may be reversible, such as, for example, via a labile moiety (e.g., via a chemical cross-linker, including chemical cross-linkers described herein). Upon application of a stimulus, the labile moiety may be cleaved and the immobilized reagent set free. In some embodiments, the labile moiety is a disulfide bond. For example, in the case where an oligonucleotide barcode is immobilized to a gel bead via a disulfide bond, exposure of the disulfide bond to a reducing agent can cleave the disulfide bond and free the oligonucleotide barcode from the bead. The labile moiety may be included as part of a gel bead or microcapsule, as part of a chemical linker that links a reagent or analyte to a gel bead or microcapsule, and/or as part of a reagent or analyte. In some embodiments, at least one bead oligonucleotide of the plurality of bead oligonucleotides can be immobilized on the particle, partially immobilized on the particle, enclosed in the particle, partially enclosed in the particle, or any combination thereof.

In some embodiments, a gel bead can comprise a wide range of different polymers including but not limited to: polymers, heat sensitive polymers, photosensitive polymers, magnetic polymers, pH sensitive polymers, salt-sensitive polymers, chemically sensitive polymers, polyelectrolytes, polysaccharides, peptides, proteins, and/or plastics. Polymers may include but are not limited to materials such as poly(N-isopropylacrylamide) (PNIPAAm), poly(styrene sulfonate) (PSS), poly(allyl amine) (PAAm), poly(acrylic acid) (PAA), poly(ethylene imine) (PEI), poly(diallyldimethyl-ammonium chloride) (PDADMAC), poly(pyrolle) (PPy), poly(vinylpyrrolidone) (PVPON), poly(vinyl pyridine) (PVP), poly(methacrylic acid) (PMAA), poly(methyl methacrylate) (PMMA), polystyrene (PS), poly(tetrahydrofuran) (PTHF), poly(phthaladehyde) (PTHF), poly(hexyl viologen) (PHV), poly(L-lysine) (PLL), poly(L-arginine) (PARG), poly(lactic-co-glycolic acid) (PLGA).

Numerous chemical stimuli can be used to trigger the disruption, dissolution, or degradation of the beads. Examples of these chemical changes may include, but are not limited to, pH-mediated changes to the bead wall, disintegration of the bead wall via chemical cleavage of crosslink bonds, triggered depolymerization of the bead wall, and bead wall switching reactions. Bulk changes may also be used to trigger disruption of the beads.

Bulk or physical changes to the beads through various stimuli also offer many advantages in designing capsules to release reagents. Bulk or physical changes occur on a macroscopic scale, in which bead rupture is the result of mechano-physical forces induced by a stimulus. These processes may include, but are not limited to pressure induced rupture, bead wall melting, or changes in the porosity of the bead wall.

Biological stimuli may also be used to trigger disruption, dissolution, or degradation of beads. Generally, biological triggers resemble chemical triggers, but many examples use biomolecules, or molecules commonly found in living systems such as enzymes, peptides, saccharides, fatty acids, nucleic acids and the like. For example, beads may comprise polymers with peptide cross-links that are sensitive to cleavage by specific proteases. More specifically, one example may comprise a microcapsule comprising GFLGK peptide cross links. Upon addition of a biological trigger such as the protease Cathepsin B, the peptide cross links of the shell well are cleaved and the contents of the beads are released. In other cases, the proteases may be heat-activated. In another example, beads comprise a shell wall comprising cellulose. Addition of the hydrolytic enzyme chitosan serves as biologic trigger for cleavage of cellulosic bonds, depolymerization of the shell wall, and release of its inner contents.

The beads may also be induced to release their contents upon the application of a thermal stimulus. A change in temperature can cause a variety changes to the beads. A change in heat may cause melting of a bead such that the bead wall disintegrates. In other cases, the heat may increase the internal pressure of the inner components of the bead such that the bead ruptures or explodes. In still other cases, the heat may transform the bead into a shrunken dehydrated state. The heat may also act upon heat-sensitive polymers within the wall of a bead to cause disruption of the bead.

Inclusion of magnetic nanoparticles to the bead wall of microcapsules may allow triggered rupture of the beads as well as guide the beads in an array. A device of this disclosure may comprise magnetic beads for either purpose. In one example, incorporation of Fe₃O₄ nanoparticles into polyelectrolyte containing beads triggers rupture in the presence of an oscillating magnetic field stimulus.

A bead may also be disrupted, dissolved, or degraded as the result of electrical stimulation. Similar to magnetic particles described in the previous section, electrically sensitive beads can allow for both triggered rupture of the beads as well as other functions such as alignment in an electric field, electrical conductivity or redox reactions. In one example, beads containing electrically sensitive material are aligned in an electric field such that release of inner reagents can be controlled. In other examples, electrical fields may induce redox reactions within the bead wall itself that may increase porosity.

A light stimulus may also be used to disrupt the beads. Numerous light triggers are possible and may include systems that use various molecules such as nanoparticles and chromophores capable of absorbing photons of specific ranges of wavelengths. For example, metal oxide coatings can be used as capsule triggers. UV irradiation of polyelectrolyte capsules coated with SiO₂ may result in disintegration of the bead wall. In yet another example, photo switchable materials such as azobenzene groups may be incorporated in the bead wall. Upon the application of UV or visible light, chemicals such as these undergo a reversible cis-to-trans isomerization upon absorption of photons. In this aspect, incorporation of photon switches results in a bead wall that may disintegrate or become more porous upon the application of a light trigger.

In some embodiments, beads can include synthetic particles associated with the plurality of barcodes. The synthetic particles can be beads. The beads can be silica gel beads, controlled pore glass beads, magnetic beads, Dynabeads, Sephadex/Sepharose beads, cellulose beads, polystyrene beads, or any combination thereof. The bead can include a polymer, a matrix, a hydrogel, a needle array device, an antibody, or any combination thereof.

As used herein, the terms “tethered,” “attached,” and “immobilized,” are used interchangeably, and can refer to covalent or non-covalent means for attaching barcodes to a solid support. Any of a variety of different solid supports can be used as solid supports for attaching pre-synthesized barcodes or for in situ solid-phase synthesis of barcode.

In some embodiments, the solid support is a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Solid supports (e.g., beads) can comprise a variety of materials including, but not limited to, paramagnetic materials (e.g., magnesium, molybdenum, lithium, and tantalum), superparamagnetic materials (e.g., ferrite (Fe₃O₄; magnetite) nanoparticles), ferromagnetic materials (e.g., iron, nickel, cobalt, some alloys thereof, and some rare earth metal compounds), ceramic, plastic, glass, polystyrene, silica, methylstyrene, acrylic polymers, titanium, latex, Sepharose, agarose, hydrogel, polymer, cellulose, nylon, or any combination thereof.

In some embodiments, the solid support (e.g., the bead to which the labels are attached) is a hydrogel bead. In some embodiments, the bead comprises hydrogel.

The size of the solid supports (e.g., beads) can vary. For example, the diameter of the bead can range from 0.1 micrometer to 50 micrometers. In some embodiments, the diameter of the bead can be, or be about, 0.1, 0.5, 1, 2, 3, 4, 5, 6, 7, 8, 9, 10, 20, 30, 40, 50 micrometers, or a number or a range between any two of these values.

Examples of solid supports (e.g., beads) can include, but are not limited to, streptavidin beads, agarose beads, magnetic beads, Dynabeads®, MACS® microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A solid support (e.g., bead) can be associated with (e.g., impregnated with) one or more first detectable moieties (e.g., quantum dots or fluorescent dyes) to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a barcode. A bead can change size, for example, due to swelling in an organic or inorganic solution. A bead can be hydrophobic. A bead can be hydrophilic. A bead can be biocompatible.

A solid support (e.g., bead) can be visualized. The solid support can comprise a visualizing tag (e.g., fluorescent dye). A bead can be etched with an identifier (e.g., a number). The identifier can be visualized through imaging the beads.

Cellular Component Binding Reagents Associated with Oligonucleotides

Some embodiments disclosed herein provide a plurality of compositions each comprising a cellular component binding reagent (such as a protein binding reagent) that is conjugated with an oligonucleotide (also referred to herein as a reagent oligonucleotide), wherein the oligonucleotide comprises a unique identifier for the cellular component binding reagent that it is conjugated with.

In some embodiments, the cellular component binding reagent is capable of specifically binding to a cellular component target. For example, a binding target of the cellular component binding reagent can be, or comprise, a carbohydrate, a lipid, a protein, an extracellular protein, a cell-surface protein, a cell marker, a B-cell receptor, a T-cell receptor, a major histocompatibility complex, a tumor antigen, a receptor, an integrin, an intracellular protein, or any combination thereof. In some embodiments, the cellular component binding reagent (e.g., a protein binding reagent) is capable of specifically binding to an antigen target or a protein target. In some embodiments, each of the reagent oligonucleotides can comprise a detection sequence. The detection sequence can bind or hybridize to the capture sequence of the bead oligonucleotide.

In some embodiments, the reagent oligonucleotide can comprise a binding site for a primer, such as universal primer. In some embodiments, the reagent oligonucleotide can comprise at least one binding site for each of two or more primers. In some embodiments, the reagent oligonucleotide can comprise at least two binding sites for a primer. The primer binding sites can be used for amplification of the reagent oligonucleotides, for example, by PCR amplification

Any suitable cellular component binding reagents are contemplated in this disclosure, such as protein binding reagents, antibodies or fragments thereof, aptamers, small molecules, ligands, peptides, oligonucleotides, etc., or any combination thereof. In some embodiments, the cellular component binding reagents can be polyclonal antibodies, monoclonal antibodies, recombinant antibodies, single chain antibody (sc-Ab), or fragments thereof, such as Fab, Fv, etc. In some embodiments, the plurality of cellular component binding reagents can comprise, or comprise about, 20, 30, 40, 50, 60, 70, 80, 90, 100, 200, 300, 400, 500, 600, 700, 800, 900, 1000, 2000, 5000, or a number or a range between any two of these values, different cellular component reagents. In some embodiments, the plurality of cellular component binding reagents can comprise at least, or comprise at most, 20, 30, 40, 50, 60, 70, 80, 90, 100, 200, 300, 400, 500, 600, 700, 800, 900, 1000, 2000, or 5000, different cellular component reagents.

The reagent oligonucleotide can be conjugated with the cellular component binding reagent through various mechanism. In some embodiments, the reagent oligonucleotide can be conjugated with the cellular component binding reagent covalently. In some embodiment, the reagent oligonucleotide can be conjugated with the cellular component binding reagent non-covalently. In some embodiments, the reagent oligonucleotide is conjugated with the cellular component binding reagent through a linker. The linker can be, for example, cleavable or detachable from the cellular component binding reagent and/or the reagent oligonucleotide. In some embodiments, the linker can comprise a chemical group that reversibly attaches the reagent oligonucleotide to the cellular component binding reagents. The chemical group can be conjugated to the linker, for example, through an amine group. In some embodiments, the linker can comprise a chemical group that forms a stable bond with another chemical group conjugated to the cellular component binding reagent. For example, the chemical group can be a UV photocleavable group, a disulfide bond, a streptavidin, a biotin, an amine, etc. In some embodiments, the chemical group can be conjugated to the cellular component binding reagent through a primary amine on an amino acid, such as lysine, or the N-terminus. Commercially available conjugation kits, such as the Protein-Oligo Conjugation Kit (Solulink, Inc., San Diego, California), the Thunder-Link® oligo conjugation system (Innova Biosciences, Cambridge, United Kingdom), etc., can be used to conjugate the reagent oligonucleotide to the cellular component binding reagent.

The reagent oligonucleotide can be conjugated to any suitable site of the cellular component binding reagent (e.g., a protein binding reagent), as long as it does not interfere with the specific binding between the cellular component binding reagent and its cellular component target. In some embodiments, the cellular component binding reagent is a protein, such as an antibody. In some embodiments, the cellular component binding reagent is not an antibody. In some embodiments, the reagent oligonucleotide can be conjugated to the antibody anywhere other than the antigen-binding site, for example, the Fc region, the C_{H}1 domain, the C_{H}2 domain, the C_{H}3 domain, the C_{L} domain, etc. Methods of conjugating reagent oligonucleotides to cellular component binding reagents (e.g., antibodies) have been previously disclosed, for example, in U.S. Pat. No. 6,531,283, the content of which is hereby expressly incorporated by reference in its entirety. Stoichiometry of reagent oligonucleotide to cellular component binding reagent can be varied. To increase the sensitivity of detecting the reagent oligonucleotide in sequencing, it may be advantageous to increase the ratio of reagent oligonucleotide to cellular component binding reagent during conjugation. In some embodiments, each cellular component binding reagent can be conjugated with a single s molecule. In some embodiments, each cellular component binding reagent can be conjugated with more than one oligonucleotide molecule, for example, at least, or at most, 2, 3, 4, 5, 10, 20, 30, 40, 50, 100, 1000, or a number or a range between any two of these values, oligonucleotide molecules wherein each of the oligonucleotide molecule comprises the same, or different, unique identifiers. In some embodiments, each cellular component binding reagent can be conjugated with more than one oligonucleotide molecule, for example, at least, or at most, 2, 3, 4, 5, 10, 20, 30, 40, 50, 100, 1000, oligonucleotide molecules, wherein each of the oligonucleotide molecule comprises the same, or different, unique identifiers. The methods and compositions provided herein can be employed in concert with the methods and compositions described in U.S. Patent Application No. 63/239,369, filed on Aug. 31, 2021, entitled “RNA PRESERVATION AND RECOVERY FROM FIXED CELLS”, the content of which is incorporated herein by reference in its entirety. The methods and compositions provided herein can be employed in concert with blocking reagents, such as those described in U.S. Patent Application No. 63/239,367, filed on Aug. 31, 2021, entitled “USE OF DECOY POLYNUCLEOTIDES IN SINGLE CELL MULTIOMICS”, the content of which is incorporated herein by reference in its entirety. The systems, methods, compositions, and kits provided herein can, in some embodiments, be employed in concert with the systems, methods, compositions, and kits described in PCT Application Publication No. WO/2021/163374, the content of which is incorporated herein by reference in its entirety.

In some embodiments, the plurality of cellular component binding reagents are capable of specifically binding to a plurality of cellular component targets in a sample, such as a single cell, a plurality of cells, a tissue sample, a tumor sample, a blood sample, or the like. In some embodiments, the plurality of cellular component targets comprises a cell-surface protein, a cell marker, a B-cell receptor, a T-cell receptor, an antibody, a major histocompatibility complex, a tumor antigen, a receptor, or any combination thereof. In some embodiments, the plurality of cellular component targets can comprise intracellular cellular components. In some embodiments, the plurality of cellular component targets can comprise intracellular cellular components. In some embodiments, the plurality of cellular components can be, or be about, 1%, 2%, 3%, 4%, 5%, 6%, 7%, 8%, 9%, 10%, 20%, 30%, 40%, 50%, 60%, 70%, 80%, 90%, 95%, 98%, 99%, or a number or a range between any two of these values, of all the cellular components (e.g., proteins) in a cell or an organism. In some embodiments, the plurality of cellular components can be at least, or be at most, 1%, 2%, 3%, 4%, 5%, 6%, 7%, 8%, 9%, 10%, 20%, 30%, 40%, 50%, 60%, 70%, 80%, 90%, 95%, 98%, or 99%, of all the cellular components (e.g., proteins) in a cell or an organism. In some embodiments, the plurality of cellular component targets can comprise, or comprise about, 2, 3, 4, 5, 10, 20, 30, 40, 50, 100, 1000, 10000, or a number or a range between any two of these values, different cellular component targets. In some embodiments, the plurality of cellular component targets can comprise at least, or comprise at most, 2, 3, 4, 5, 10, 20, 30, 40, 50, 100, 1000, 10000, different cellular component targets.

FIG. 2 shows a schematic illustration of an exemplary cellular component binding reagent (e.g., an antibody) that is associated (e.g., conjugated) with an oligonucleotide comprising a unique identifier sequence for the antibody. An oligonucleotide-conjugated with a cellular component binding reagent, an oligonucleotide for conjugation with a cellular component binding reagent, or an oligonucleotide previously conjugated with a cellular component binding reagent can be referred to herein as an antibody oligonucleotide. An oligonucleotide-conjugated with an antibody, an oligonucleotide for conjugation with an antibody, or an oligonucleotide previously conjugated with an antibody can be referred to herein as an antibody oligonucleotide (abbreviated as an “AbOligo” or “AbO”). The oligonucleotide can comprise one or more linker, one or more unique identifier for the antibody, and one or more detection sequences that can bind (or hybridize to) that detection oligonucleotides (or molecules of a detection oligonucleotide).

In some embodiments, the reagent oligonucleotide (e.g., the sample oligonucleotide) comprises a nucleotide sequence of, of about, or at least, or at least about, or at most, or of at most about, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or a number or a range between any two of these values, nucleotides in length.

In some embodiments, the cellular component binding reagent comprises an antibody, a tetramer, an aptamer, a protein scaffold, or a combination thereof. The binding reagent oligonucleotide can be conjugated to the cellular component binding reagent, for example, through a linker. The binding reagent oligonucleotide can comprise the linker. The linker can comprise a chemical group. The chemical group can be reversibly, or irreversibly, attached to the molecule of the cellular component binding reagent. The chemical group can be selected from a UV photocleavable group, a disulfide bond, a streptavidin, a biotin, an amine, and any combination thereof.

In some embodiments, the cellular component binding reagent can bind to ADAM10, CD156c, ANO6, ATP1B2, ATP1B3, BSG, CD147, CD109, CD230, CD29, CD298, ATP1B3, CD44, CD45, CD47, CD51, CD59, CD63, CD97, CD98, SLC3A2, CLDND1, HLA-ABC, ICAM1, ITFG3, MPZL1, NA K ATPase alpha1, ATP1A1, NPTN, PMCA ATPase, ATP2B1, SLC1A5, SLC29A1, SLC2A1, SLC44A2, or any combination thereof.

In some embodiments, the protein target is, or comprises, an extracellular protein, an intracellular protein, or any combination thereof. In some embodiments, the antigen or protein target is, or comprises, a cell-surface protein, a cell marker, a B-cell receptor, a T-cell receptor, a major histocompatibility complex, a tumor antigen, a receptor, an integrin, or any combination thereof. The antigen or cellular component can be, or comprise, a lipid, a carbohydrate, or any combination thereof. The protein target can be selected from a group comprising a number of protein targets. The number of cellular components can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, or a number or a range between any two of these values. The number of protein targets can be at least, or be at most, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000.

The cellular component binding reagent (e.g., a protein binding reagent) can be associated with two or more reagent oligonucleotide with an identical sequence. The cellular component binding reagent can be associated with two or more reagent oligonucleotides with different sequences. The number of reagent oligonucleotides associated with the cellular component binding reagent can be different in different implementations. In some embodiments, the number of reagent oligonucleotides, whether having an identical sequence, or different sequences, can be, be about, be at last, be at least about, be at most, or be at most about, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values.

### Detectable Moieties

In some embodiments, a detectable moiety (e.g., a first detectable moiety, a second detectable moiety, a bead dye, a detection dye, or precursors thereof) comprises an optical moiety, a luminescent moiety, an electrochemically active moiety, a nanoparticle, or a combination thereof. In some embodiments, the luminescent moiety comprises a chemiluminescent moiety, an electroluminescent moiety, a photoluminescent moiety, or a combination thereof. In some embodiments, the photoluminescent moiety comprises a fluorescent moiety, a phosphorescent moiety, or a combination thereof. In some embodiments, the fluorescent moiety comprises a fluorescent dye. In some embodiments, the nanoparticle comprises a quantum dot. In some embodiments, the methods comprise performing a reaction to convert the detectable moiety precursor into the detectable moiety. In some embodiments, performing a reaction to convert the detectable moiety precursor into the detectable moiety comprises contacting the detectable moiety precursor with a substrate. In some such embodiments, contacting the detectable moiety precursor with a substrate yields a detectable byproduct of a reaction between the two molecules.

### Detectable Moiety Properties and Structures

In some embodiments, detectable labels, moieties, or markers can be detectible based on, for example, fluorescence emission, absorbance, fluorescence polarization, fluorescence lifetime, fluorescence wavelength, absorbance wavelength, Stokes shift, light scatter, mass, molecular mass, redox, acoustic, Raman, magnetism, radio frequency, enzymatic reactions (including chemiluminescence and electro-chemiluminescence) or combinations thereof. For example, the label may be a fluorophore, a chromophore, an enzyme, an enzyme substrate, a catalyst, a redox label, a radio label, an acoustic label, a Raman (SERS) tag, a mass tag, an isotope tag (e.g., isotopically pure rare earth element), a magnetic particle, a microparticle, a nanoparticle, an oligonucleotide, or any combination thereof. In some embodiments, the label is a fluorophore (i.e., a fluorescent label, fluorescent dye, etc.). Fluorophores of interest may include but are not limited to dyes suitable for use in analytical applications (e.g., flow cytometry, imaging, etc.), such as an acridine dye, anthraquinone dyes, arylmethane dyes, diarylmethane dyes (e.g., diphenyl methane dyes), chlorophyll containing dyes, triarylmethane dyes (e.g., triphenylmethane dyes), azo dyes, diazonium dyes, nitro dyes, nitroso dyes, phthalocyanine dyes, cyanine dyes, asymmetric cyanine dyes, quinon-imine dyes, azine dyes, eurhodin dyes, safranin dyes, indamins, indophenol dyes, fluorine dyes, oxazine dye, oxazone dyes, thiazine dyes, thiazole dyes, xanthene dyes, fluorene dyes, pyronin dyes, fluorine dyes, rhodamine dyes, phenanthridine dyes, as well as dyes combining two or more of the aforementioned dyes (e.g., in tandem), polymeric dyes having one or more monomeric dye units and mixtures of two or more of the aforementioned dyes thereof. A large number of dyes are commercially available from a variety of sources, such as, for example, Molecular Probes (Eugene, OR), Dyomics GmbH (Jena, Germany), Sigma-Aldrich (St. Louis, MO), Sirigen, Inc. (Santa Barbara, CA) and Exciton (Dayton, OH). For example, the fluorophore may include 4-acetamido-4′-isothiocyanatostilbene-2,2′disulfonic acid; acridine and derivatives such as acridine, acridine orange, acridine yellow, acridine red, and acridine isothiocyanate; allophycocyanin, phycoerythrin, peridinin-chlorophyll protein, 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy3.5, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; dye-conjugated polymers (i.e., polymer-attached dyes) such as fluorescein isothiocyanate-dextran as well as dyes combining two or more dyes (e.g., in tandem), polymeric dyes having one or more monomeric dye units and mixtures of two or more of the aforementioned dyes or combinations thereof.

The detectable moiety can be selected from a group of spectrally-distinct detectable moieties. Spectrally-distinct detectable moieties include detectable moieties with distinguishable emission spectra even if their emission spectral may overlap. Non-limiting examples of detectable moieties include Xanthene derivatives: fluorescein, rhodamine, Oregon green, eosin, and Texas red; Cyanine derivatives: cyanine, indocarbocyanine, oxacarbocyanine, thiacarbocyanine, and merocyanine; Squaraine derivatives and ring-substituted squaraines, including Seta, SeTau, and Square dyes; Naphthalene derivatives (dansyl and prodan derivatives); Coumarin derivatives; oxadiazole derivatives: pyridyloxazole, nitrobenzoxadiazole and benzoxadiazole; Anthracene derivatives: anthraquinones, including DRAQ5, DRAQ7 and CyTRAK Orange; Pyrene derivatives: cascade blue; Oxazine derivatives: Nile red, Nile blue, cresyl violet, oxazine 170; Acridine derivatives: proflavin, acridine orange, acridine yellow; Arylmethine derivatives: auramine, crystal violet, malachite green; and Tetrapyrrole derivatives: porphin, phthalocyanine, bilirubin. Other non-limiting examples of detectable moieties include Hydroxycoumarin, Aminocoumarin, Methoxycoumarin, Cascade Blue, Pacific Blue, Pacific Orange, Lucifer yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhodamine B, Texas Red, Allophycocyanin (APC), APC-Cy7 conjugates, Hoechst 33342, DAPI, Hoechst 33258, SYTOX Blue, Chromomycin A3, Mithramycin, YOYO-1, Ethidium Bromide, Acridine Orange, SYTOX Green, TOTO-1, TO-PRO-1, TO-PRO: Cyanine Monomer, Thiazole Orange, CyTRAK Orange, Propidium Iodide (PI), LDS 751, 7-AAD, SYTOX Orange, TOTO-3, TO-PRO-3, DRAQ5, DRAQ7, Indo-1, Fluo-3, Fluo-4, DCFH, DHR, and SNARF.

In some embodiments, fluorophores of interest may include, but are not limited to, dyes suitable for use in analytical applications (e.g., flow cytometry, imaging, etc.), such as an acridine dye, anthraquinone dyes, arylmethane dyes, diarylmethane dyes (e.g., diphenyl methane dyes), chlorophyll containing dyes, triarylmethane dyes (e.g., triphenylmethane dyes), azo dyes, diazonium dyes, nitro dyes, nitroso dyes, phthalocyanine dyes, cyanine dyes, asymmetric cyanine dyes, quinon-imine dyes, azine dyes, eurhodin dyes, safranin dyes, indamins, indophenol dyes, fluorine dyes, oxazine dye, oxazone dyes, thiazine dyes, thiazole dyes, xanthene dyes, fluorene dyes, pyronin dyes, fluorine dyes, rhodamine dyes, phenanthridine dyes, as well as dyes combining two or more dyes (e.g., in tandem) as well as polymeric dyes having one or more monomeric dye units, as well as mixtures of two or more dyes thereof. For example, the fluorophore may be 4-acetamido-4′-isothiocyanatostilbene-2,2′disulfonic acid; acridine and derivatives such as acridine, acridine orange, acridine yellow, acridine red, and acridine isothiocyanate; allophycocyanin, phycoerythrin, peridinin-chlorophyll protein, 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; dye-conjugated polymers (i.e., polymer-attached dyes) such as fluorescein isothiocyanate-dextran as well as dyes combining two or more of the aforementioned dyes (e.g., in tandem), polymeric dyes having one or more monomeric dye units and mixtures of two or more of the aforementioned dyes thereof.

The group of spectrally distinct detectable moieties can, for example, include five different fluorophores, five different chromophores, a combination of five fluorophores and chromophores, a combination of four different fluorophores and a non-fluorophore, a combination of four chromophores and a non-chromophore, or a combination of four fluorophores and chromophores and a non-fluorophore non-chromophore. In some embodiments, the detectable moieties can be one of 1, 2, 3, 4, 5, 6, 7, 8, 9, 10, 20, 30, 40, 50, 60, 70, 80, 90, 100, 1000, 2000, 3000, 4000, 5000, 6000, 7000, 8000, 9000, 10000, or a number or a range between any two of these values, of spectrally-distinct moieties.

The excitation wavelength of the detectable moieties can vary, for example be, or be about, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 nanometers, or a number or a range between any two of these values. The emission wavelength of the detectable moieties can also vary, for example be, or be about, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 nanometers, or a number or a range between any two of these values.

The molecular weights of the detectable moieties can vary, for example be, or be about, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 Daltons (Da), or a number or a range between any two of these values. The molecular weights of the detectable moieties can also vary, for example be, or be about, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 kilo Daltons (kDa), or a number or a range between any two of these values.

### Polymeric Dyes

In some instances, the fluorophore (i.e., dye) is a fluorescent polymeric dye. Fluorescent polymeric dyes that find use in the subject methods and systems can vary. In some instances of the method, the polymeric dye includes a conjugated polymer.

Conjugated polymers (CPs) are characterized by a delocalized electronic structure which includes a backbone of alternating unsaturated bonds (e.g., double and/or triple bonds) and saturated (e.g., single bonds) bonds, where 71-electrons can move from one bond to the other. As such, the conjugated backbone may impart an extended linear structure on the polymeric dye, with limited bond angles between repeat units of the polymer. For example, proteins and nucleic acids, although also polymeric, in some cases do not form extended-rod structures but rather fold into higher-order three-dimensional shapes. In addition, CPs may form “rigid-rod” polymer backbones and experience a limited twist (e.g., torsion) angle between monomer repeat units along the polymer backbone chain. In some instances, the polymeric dye includes a CP that has a rigid rod structure. As summarized above, the structural characteristics of the polymeric dyes can have an effect on the fluorescence properties of the molecules.

Any convenient polymeric dye may be utilized in the subject methods and systems. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some embodiments, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “excitation light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via energy transfer.

In some instances the polymer may be used as a direct fluorescent reporter, for example fluorescent polymers having high extinction coefficients, high brightness, etc. In some instances, the polymer may be used as a strong chromophore where the color or optical density is used as an indicator.

Polymeric dyes of interest include, but are not limited to, those dyes described by Gaylord et al. in US Publication Nos. 20040142344, 20080293164, 20080064042, 20100136702, 20110256549, 20120028828, 20120252986, 20130190193 and 20160025735 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety.

In some embodiments, the polymeric dye includes a conjugated polymer including a plurality of first optically active units forming a conjugated system, having a first absorption wavelength (e.g., as described herein) at which the first optically active units absorb light to form an excited state. The conjugated polymer (CP) may be polycationic, polyanionic and/or a charge-neutral conjugated polymer.

The CPs may be water soluble for use in biological samples. Any convenient substituent groups may be included in the polymeric dyes to provide for increased water-solubility, such as a hydrophilic substituent group, e.g., a hydrophilic polymer, or a charged substituent group, e.g., groups that are positively or negatively charged in an aqueous solution, e.g., under physiological conditions. Any convenient water-soluble groups (WSGs) may be utilized in the subject light harvesting multichromophores. The term “water-soluble group” refers to a functional group that is well solvated in aqueous environments and that imparts improved water solubility to the molecules to which it is attached. In some embodiments, a WSG increases the solubility of the multichromophore in a predominantly aqueous solution (e.g., as described herein), as compared to a multichromophore which lacks the WSG. The water-soluble groups may be any convenient hydrophilic group that is well solvated in aqueous environments. In some embodiments, the hydrophilic water-soluble group is charged, e.g., positively or negatively charged or zwitterionic. In some embodiments, the hydrophilic water-soluble group is a neutral hydrophilic group. In some embodiments, the WSG is a hydrophilic polymer, e.g., a polyethylene glycol, a cellulose, a chitosan, or a derivative thereof.

As used herein, the terms “polyethylene oxide”, “PEO”, “polyethylene glycol” and “PEG” are used interchangeably and refer to a polymer including a chain described by the formula —(CH₂—CH₂—O—)ₙ— or a derivative thereof. In some embodiments, “n” is 5000 or less, such as 1000 or less, 500 or less, 200 or less, 100 or less, 50 or less, 40 or less, 30 or less, 20 or less, 15 or less, such as 5 to 15, or 10 to 15. It is understood that the PEG polymer may be of any convenient length and may include a variety of terminal groups, including but not limited to, alkyl, aryl, hydroxyl, amino, acyl, acyloxy, and amido terminal groups. Functionalized PEGs that may be adapted for use in the subject multichromophores include those PEGs described by S. Zalipsky in “Functionalized poly(ethylene glycol) for preparation of biologically relevant conjugates”, Bioconjugate Chemistry 1995, 6 (2), 150-165. Water soluble groups of interest include, but are not limited to, carboxylate, phosphonate, phosphate, sulfonate, sulfate, sulfinate, ester, polyethylene glycols (PEG) and modified PEGs, hydroxyl, amine, ammonium, guanidinium, polyamine and sulfonium, polyalcohols, straight chain or cyclic saccharides, primary, secondary, tertiary, or quaternary amines and polyamines, phosphonate groups, phosphinate groups, ascorbate groups, glycols, including, polyethers, —COOM′, —SO₃M′, —PO₃M′, —NR₃⁺, Y′, (CH₂CH₂O)ₚR and mixtures thereof, where Y′ can be any halogen, sulfate, sulfonate, or oxygen containing anion, p can be 1 to 500, each R can be independently H or an alkyl (such as methyl) and M′ can be a cationic counterion or hydrogen, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, and polyethylene glycol, wherein yy is selected from 1 to 1000, X is selected from 0, S, and NR^{ZZ} and R^{ZZ} and R^{YY} are independently selected from H and C1-3 alkyl.

The polymeric dye may have any convenient length. In some embodiments, the particular number of monomeric repeat units or segments of the polymeric dye may fall within the range of 2 to 500,000, such as 2 to 100,000, 2 to 30,000, 2 to 10,000, 2 to 3,000 or 2 to 1,000 units or segments, or such as 100 to 100,000, 200 to 100,000, or 500 to 50,000 units or segments. In some embodiments, the number of monomeric repeat units or segments of the polymeric dye is within the range of 2 to 1000 units or segments, such as from 2 to 750 units or segments, such as from 2 to 500 units or segments, such as from 2 to 250 units or segment, such as from 2 to 150 units or segment, such as from 2 to 100 units or segments, such as from 2 to 75 units or segments, such as from 2 to 50 units or segments and including from 2 to 25 units or segments.

The polymeric dyes may be of any convenient molecular weight (MW). In some embodiments, the MW of the polymeric dye may be expressed as an average molecular weight. In some instances, the polymeric dye has an average molecular weight of from 500 to 500,000, such as from 1,000 to 100,000, from 2,000 to 100,000, from 10,000 to 100,000 or even an average molecular weight of from 50,000 to 100,000. In some embodiments, the polymeric dye has an average molecular weight of 70,000.

In some embodiments, the polymeric dye includes the following structure:

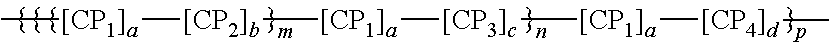

wherein CP₁, CP₂, CP₃ and CP₄ are independently a conjugated polymer segment or an oligomeric structure, wherein one or more of CP₁, CP₂, CP₃ and CP₄ are bandgap-modifying n-conjugated repeat units.

In some embodiments, the conjugated polymer is a polyfluorene conjugated polymer, a polyphenylene vinylene conjugated polymer, a polyphenylene ether conjugated polymer, a polyphenylene polymer, among other types of conjugated polymers.

In some instances, the polymeric dye includes the following structure:

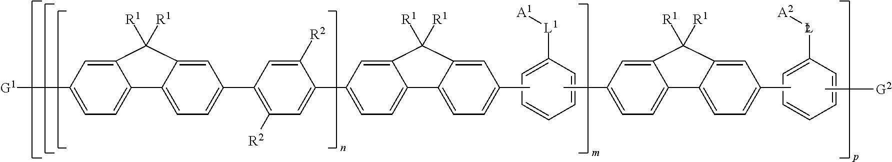

wherein each R¹ is independently a solubilizing group or a linker-dye; L¹ and L² are optional linkers; each R2 is independently H or an aryl substituent; each A¹ and A² is independently H, an aryl substituent or a fluorophore; G¹ and G² are each independently selected from a terminal group, a πconjugated segment, a linker and a linked specific binding member; each n and each m are independently 0 or an integer from 1 to 10,000; and p is an integer from 1 to 100,000. Solubilizing groups of interest include, but is not limited to a water-soluble functional group such as a hydrophilic polymer (e.g., polyalkylene oxide, cellulose, chitosan, etc.), as well as alkyl, aryl and heterocycle groups further substituted with a hydrophilic group such as a polyalkylene oxide (e.g., polyethylglycol including a PEG of 2-20 units), an ammonium, a sulphonium, a phosphonium, as well has a charged (positively, negatively or zwitterionic) hydrophilic water soluble group and the like.

In some embodiments, the polymeric dye includes, as part of the polymeric backbone, a conjugated segment having one of the following structures:

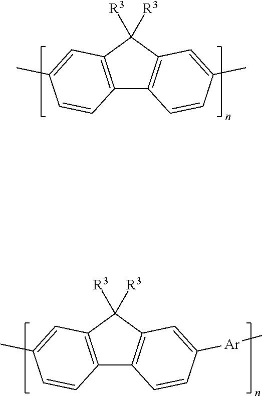

where each R³ is independently an optionally substituted wat-soluble functional group such as a hydrophilic polymer (e.g., polyalkylene oxide, cellulose, chitosan, etc.) or an alkyl or aryl group further substituted with a hydrophilic group such as a polyalkylene oxide (e.g., polyethylglycol including a PEG of 2-20 units), an ammonium, a sulphonium, a phosphonium, as well has a charged (positively, negatively or zwitterionic) hydrophilic water soluble group; Ar is an optionally substituted aryl or heteroaryl group; and n is 1 to 10000. In some embodiments, R3 is an optionally substituted alkyl group. In some embodiments, R3 is an optionally substituted aryl group. In some embodiments, R³ is substituted with a polyethyleneglycol, a dye, a chemoselective functional group or a specific binding moiety. In some embodiments, Ar is substituted with a polyethyleneglycol, a dye, a chemoselective functional group or a specific binding moiety.

In some embodiments, the polymeric dye includes the following structure:

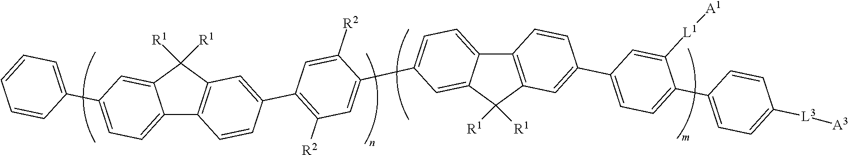

wherein each R¹ is a solubilizing group or a linker dye group; each R² is independently H or an aryl substituent; L₁ and L₂ are optional linkers; each A1 and A3 are independently H, a fluorophore, a functional group or a specific binding moiety (e.g., an antibody); and n and m are each independently 0 to 10000, wherein n+m>1.

The polymeric dye may have one or more desirable spectroscopic properties, such as a particular absorption maximum wavelength, a particular emission maximum wavelength, extinction coefficient, quantum yield, and the like (see e.g., Chattopadhyay et al., “Brilliant violet fluorophores: A new class of ultrabright fluorescent compounds for immunofluorescence experiments.” Cytometry Part A, 81A(6), 456-466, 2012).

In some embodiments, the polymeric dye has an absorption curve between 280 and 850 nm. In some embodiments, the polymeric dye has an absorption maximum in the range 280 and 850 nm. In some embodiments, the polymeric dye absorbs incident light having a wavelength in the range between 280 and 850 nm, where specific examples of absorption maxima of interest include, but are not limited to: 348 nm, 355 nm, 405 nm, 407 nm, 445 nm, 488 nm, 640 nm and 652 nm. In some embodiments, the polymeric dye has an absorption maximum wavelength in a range selected from 280-310 nm, 305-325 nm, 320-350 nm, 340-375 nm, 370-425 nm, 400-450 nm, 440-500 nm, 475-550 nm, 525-625 nm, 625-675 nm and 650-750 nm. In some embodiments, the polymeric dye has an absorption maximum wavelength of 348 nm, 355 nm, 405 nm, 407 nm, 445 nm, 488 nm, 640 nm, 652 nm, or a range between any two of these values.

In some embodiments, the polymeric dye has an emission maximum wavelength ranging from 400 to 850 nm, such as 415 to 800 nm, where specific examples of emission maxima of interest include, but are not limited to: 395 nm, 421 nm, 445 nm, 448 nm, 452 nm, 478 nm, 480 nm, 485 nm, 491 nm, 496 nm, 500 nm, 510 nm, 515 nm, 519 nm, 520 nm, 563 nm, 570 nm, 578 nm, 602 nm, 612 nm, 650 nm, 661 nm, 667 nm, 668 nm, 678 nm, 695 nm, 702 nm, 711 nm, 719 nm, 737 nm, 785 nm, 786 nm, 805 nm. In some embodiments, the polymeric dye has an emission maximum wavelength in a range selected from 380-400 nm, 410-430 nm, 470-490 nm, 490-510 nm, 500-520 nm, 560-580 nm, 570-595 nm, 590-610 nm, 610-650 nm, 640-660 nm, 650-700 nm, 700-720 nm, 710-750 nm, 740-780 nm and 775-795 nm. In some embodiments, the polymeric dye has an emission maximum of 395 nm, 421 nm, 478 nm, 480 nm, 485 nm, 496 nm, 510 nm, 570 nm, 602 nm, 650 nm, 711 nm, 737 nm, 750 nm, 786 nm, or a range of any two of these values. In some embodiments, the polymeric dye has an emission maximum wavelength of 421 nm 5 nm, 510 nm±5 nm, 570 nm±5 nm, 602 nm±5 nm, 650 nm±5 nm, 711 nm±5 nm, 786 nm 5 nm, or a range of any two of these values. In some embodiments, the polymeric dye has an emission maximum selected from 421 nm, 510 nm, 570 nm, 602 nm, 650 nm, 711 nm and 786 nm.

In some embodiments, the polymeric dye has an extinction coefficient of 1×106 cm−1M−1 or more, such as 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In some embodiments, the polymeric dye has a quantum yield of 0.05 or more, such as 0.1 or more, 0.15 or more, 0.2 or more, 0.25 or more, 0.3 or more, 0.35 or more, 0.4 or more, 0.45 or more, 0.5 or more, 0.6 or more, 0.7 or more, 0.8 or more, 0.9 or more, 0.95 or more, 0.99 or more and including 0.999 or more. For example, the quantum yield of polymeric dyes of interest may range from 0.05 to 1, such as from 0.1 to 0.95, such as from 0.15 to 0.9, such as from 0.2 to 0.85, such as from 0.25 to 0.75, such as from 0.3 to 0.7 and including a quantum yield of from 0.4 to 0.6. In some embodiments, the polymeric dye has a quantum yield of 0.1 or more. In some embodiments, the polymeric dye has a quantum yield of 0.3 or more. In some embodiments, the polymeric dye has a quantum yield of 0.5 or more. In some embodiments, the polymeric dye has a quantum yield of 0.6 or more. In some embodiments, the polymeric dye has a quantum yield of 0.7 or more. In some embodiments, the polymeric dye has a quantum yield of 0.8 or more. In some embodiments, the polymeric dye has a quantum yield of 0.9 or more. In some embodiments, the polymeric dye has a quantum yield of 0.95 or more. In some embodiments, the polymeric dye has an extinction coefficient of 1×10⁶ or more and a quantum yield of 0.3 or more. In some embodiments, the polymeric dye has an extinction coefficient of 2×106 or more and a quantum yield of 0.5 or more.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of cellular component quantification comprising:
contacting one or more cells of each of a plurality of samples with a plurality of cellular component binding reagents each associated with a reagent oligonucleotide, wherein two of the plurality of cellular component binding reagents are capable of binding to two different cellular components, or two different regions of a cellular component, and wherein each reagent oligonucleotide comprises (i) a reagent-specific sequence specific to the cellular component binding reagent associated thereto and (ii) a detection sequence, to obtain cells comprising cellular components bound to cellular component binding reagents of the plurality of cellular component binding reagents;
removing cellular component binding reagents of the plurality of cellular component binding reagents not bound to the cells;
contacting reagent oligonucleotides associated with cellular component binding reagents of the plurality of cellular component binding reagents not removed with a plurality of solid supports, wherein each of the plurality of solid supports is associated with one or more first detectable moieties, or precursors thereof, and comprises a plurality of solid support oligonucleotides, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences, to obtain reagent oligonucleotides bound to the plurality of solid supports;
contacting the reagent oligonucleotides bound to the plurality of solid supports with a detection oligonucleotide associated with one or more second detectable moieties, or precursors thereof and comprising a binding sequence capable of binding to the detection sequences of the reagent oligonucleotides to obtain reagent oligonucleotides bound to the plurality of solid supports and the detection oligonucleotide; and
detecting the one or more first detectable moieties and the one or more second detectable moieties for each of the plurality of solid supports to determine the identity and the quantity, respectively, of each of the cellular components for each of the plurality of samples.

2. A method of cellular component quantification comprising:
(a) providing one or more cells from each of a plurality of samples and with cellular components bound to cellular component binding reagents of a plurality of cellular component binding reagents (1) capable of binding to different cellular components, or regions thereof, and (2) each associated with a reagent oligonucleotide comprising (i) a reagent-specific sequence specific to the cellular component binding reagent associated thereto, and (ii) a detection sequence; and
for each of the plurality of samples:
(b) contacting reagent oligonucleotides, associated with or previously associated with the cellular component binding reagents bound to the cellular components of or from the cells of the sample, with a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of the reagent oligonucleotides, thereby obtaining reagent oligonucleotides bound to the plurality of solid supports;
(c) contacting the reagent oligonucleotides bound to the plurality of solid supports with a detection oligonucleotide associated with one or more second detectable moieties, or precursors thereof, and comprising a binding sequence capable of binding to the detection sequences of the reagent oligonucleotides, thereby obtaining reagent oligonucleotides bound to both the plurality of solid supports and the detection oligonucleotide; and
(d) detecting the one or more first detectable moieties and the one or more second detectable moieties for each of the plurality of solid supports to determine the identity and the quantity, respectively, of each of the cellular components for each of the plurality of samples.

3. The method of any one of claims 1-2, wherein determining the identity and the quantity of each of the cellular components for each of the plurality of samples comprises:
detecting the presence and/or amount of the one or more first detectable moieties and the one or more second detectable moieties for each of the plurality of solid supports, wherein the presence and/or amount of the one or more first detectable moieties and the presence and/or amount of the one or more second detectable moieties determined for a solid support indicate the identity and the quantity, respectively, of each of the cellular components for each of the plurality of samples.

4. The method of any one of claims 1-3, wherein detecting the presence and/or amount of the one or more first detectable moieties and the one or more second detectable moieties comprises:
measuring emissions of the one or more first detectable moieties and the one or more second detectable moieties with an instrument, optionally measuring emissions using flow cytometry, optionally wherein the flow cytometry comprises fluorescence-activated cell sorting (FACS).

5. The method of any one of claims 1-4, wherein one or more of the first detectable moieties and/or the second detectable moieties comprise an optical moiety, a luminescent moiety, an electrochemically active moiety, a nanoparticle, or a combination thereof.

6. The method of any one of claims 1-5, wherein the luminescent moiety comprises a chemiluminescent moiety, an electroluminescent moiety, a photoluminescent moiety, or a combination thereof.

7. The method of any one of claims 1-6, wherein the photoluminescent moiety comprises a fluorescent moiety, a phosphorescent moiety, or a combination thereof.

8. The method of any one of claims 1-7, wherein the fluorescent moiety comprises a fluorescent dye.

9. The method of any one of claims 1-8, wherein the nanoparticle comprises a quantum dot.

10. The method of any one of claims 1-9, comprising performing a reaction to convert the detectable moiety precursor into the detectable moiety.

11. The method of any one of claims 1-10, further comprising:
contacting two or more solid supports with two or more predetermined concentrations of a cellular component binding reagent, wherein each of the two or more solid supports is contacted with a different predetermined concentration of the cellular component binding reagent;
contacting the two or more solid supports with the reagent oligonucleotides; and
measuring emissions of the one or more second detectable moieties of each of the two or more first solid supports with an instrument to generate a calibration curve relating the quantity of at least one cellular component to emissions of the one or more second detectable moieties.

12. The method of any one of claims 1-11, wherein the instrument comprises a flow cytometer.

13. The method of any one of claims 1-12, wherein the flow cytometer comprises a conventional flow cytometer, a spectral flow cytometer, a hyperspectral flow cytometer, an imaging flow cytometer, or any combination thereof.

14. The method of any one of claims 1-13, wherein contacting one or more cells of each of a plurality of samples with a plurality of cellular component binding reagents comprises:
partitioning the plurality of samples to a plurality of partitions, wherein a partition of the plurality of partitions comprises a single sample of the plurality of samples; and
contacting one or more cells of each of a plurality of samples with a plurality of cellular component binding reagents.

15. The method of any one of claims 1-14, wherein contacting one or more cells of each of a plurality of samples with a plurality of cellular component binding reagents comprises:
contacting one or more cells of each of a plurality of samples with a plurality of cellular component binding reagents; and
partitioning the plurality of samples to a plurality of partitions, wherein a partition of the plurality of partitions comprises a single sample of the plurality of samples.

16. The method of any one of claims 1-15, wherein providing one or more cells from each of a plurality of samples and with cellular components bound to cellular component binding reagents of a plurality of cellular component binding reagents comprises:
providing a plurality of partitions each comprising a sample of the plurality of samples, wherein a partition of the plurality of partitions comprises a single sample of the plurality of samples.

17. The method of any one of claims 1-16, wherein the plurality of samples are partitioned to the plurality of partitions prior to contacting the one or more cells of each of a plurality of samples with a plurality of cellular component binding reagents, wherein a partition of the plurality of partitions comprises a single sample of the plurality of samples.

18. The method of any one of claims 1-17, wherein the partition is a well or a droplet.

19. The method of any one of claims 1-18, wherein the plurality of partitions comprises wells of a well array, wherein the well array comprises at least about 10 to 100 wells.

20. The method of any one of claims 1-19, wherein the instrument comprises a fluorescence microscope.

21. The method of any one of claims 1-20, wherein the instrument comprises an imaging system.

22. The method of any one of claims 1-21, wherein measuring emissions of each detectable moiety of each first solid support comprises imaging the plurality of partitions.

23. The method of any one of claims 1-22, wherein the plurality of partitions are imaged sequentially.

24. The method of any one of claims 1-23, wherein the plurality of partitions are imaged simultaneously.

25. The method of any one of claims 1-24, wherein imaging comprises microscopy, confocal microscopy, time-lapse imaging microscopy, fluorescence microscopy, multi-photon microscopy, quantitative phase microscopy, surface enhanced Raman spectroscopy, videography, manual visual analysis, automated visual analysis, or any combination thereof.

26. The method of any one of claims 1-25, wherein detecting the one or more first detectable moieties and the one or more second detectable moieties for each of the plurality of solid supports comprises:
detecting the one or more first detectable moieties and the one or more second detectable moieties for each of the plurality of solid supports of each sample of the plurality of samples separately, thereby determining the identity and the quantity, respectively, of each of the cellular components for each sample of the plurality of samples,
optionally detecting the one or more first detectable moieties and the one or more second detectable moieties for each of the plurality of solid supports of each sample of the plurality of samples separately comprises detecting the one or more first detectable moieties and the one or more second detectable moieties for each of the plurality of solid supports of each partition separately.

27. The method of any one of claims 1-26, wherein the one or more first detectable moieties of the plurality of solid supports situated in each partition are predetermined, wherein said predetermined one or more first detectable moieties are distinct to each partition, and wherein detecting the one or more first detectable moieties and the one or more second detectable moieties for each of the plurality of solid supports comprises:
detecting the predetermined one or more first detectable moieties and the one or more second detectable moieties for each of the plurality of solid supports of each sample of the plurality of samples concurrently; and
associating the detected predetermined one or more first detectable moieties of each of the solid supports with the partition from which said solid support derived, thereby determining the identity and the quantity, respectively, of each of the cellular components for each sample of the plurality of samples.

28. The method of any one of claims 1-27, further comprising pooling the solid supports from each partition of the plurality of partitions, optionally the pooling is performed using a magnetic field.

29. The method of any one of claims 1-28, wherein contacting the reagent oligonucleotides bound to the plurality of solid supports with the detection oligonucleotide comprises: contacting the reagent oligonucleotides bound to the plurality of solid supports with two or more detection oligonucleotides each associated with one or more second detectable moieties, optionally wherein the two or more detection oligonucleotides are associated with an identical second detectable moieties, optionally wherein the two or more detection oligonucleotides are associated with different second detectable moieties, optionally wherein the two or more detection oligonucleotides comprise an identical binding sequence, and/or optionally wherein the two or more detection oligonucleotides comprise different binding sequences.

30. The method of any one of claims 1-29, wherein each of the plurality of solid supports is associated with two distinct first detectable moieties, and wherein two solid supports of the plurality of solid supports comprise different types and/or quantities of the two distinct first detectable moieties.

31. The method of any one of claims 1-30, comprising isolating one or more populations of interest from a starting population to obtain the plurality of samples, wherein each of the samples is a population of interest, optionally two or more of the samples of the plurality of samples comprise phenotypically different populations of interest.

32. The method of any one of claims 1-31, wherein isolating one or more populations of interest from a starting population comprises flow cytometry, optionally wherein the flow cytometry comprises fluorescence-activated cell sorting (FACS).

33. The method of any one of claims 1-32, wherein providing the cells comprises: contacting the cells of each of the plurality of samples with the plurality of cellular component binding reagents to obtain the cells with the cellular components bound to the cellular component binding reagents.

34. The method of any one of claims 1-33, wherein providing the cells comprises: removing cellular component binding reagents of the plurality of cellular component binding reagents not bound to the cells to obtain the cells with the cellular components bound to the cellular component binding reagents, optionally wherein removing the cellular component binding reagents not bound to the cells comprises washing the cells with a washing buffer.

35. The method of any one of claims 1-34, comprising permeabilizing and/or fixating the cells prior to contacting the cells of each of the plurality of samples with the plurality of cellular component binding reagents.

36. The method of any one of claims 1-35, wherein two of the plurality of cellular component binding reagents are capable of binding to two different cellular components, and/or wherein two of the plurality of cellular component binding reagents are capable of binding to two different regions of a cellular component.

37. The method of any one of claims 1-36, comprises: isolating the one or more cells from the sample, optionally wherein isolating the one or more cells comprises isolating the one or more cells from the sample using flow cytometry, optionally wherein the flow cytometry comprises fluorescence-activated cell sorting (FACS).

38. The method of any one of claims 1-37, comprising: lysing the cells, prior to contacting the reagent oligonucleotides with the plurality of solid supports.

39. The method of any one of claims 1-38, comprising: dissociating the reagent oligonucleotides from the cellular component binding reagents bound to or previously bound to the cellular components of or from the cells of the sample, prior to contacting the reagent oligonucleotides with the plurality of solid supports, optionally wherein dissociating the reagent oligonucleotides comprises: detaching the reagent oligonucleotides from the cellular component binding reagents bound to or previously bound to the cellular components of or from the cells of the sample by UV photocleaving, chemical treatment, heat treatment, enzyme treatment, or a combination thereof.

40. The method of any one of claims 1-39, wherein the cellular components comprise a protein, a lipid, a carbohydrate, or a combination thereof, and/or wherein the cellular components comprise an extracellular cellular component, a cell surface cellular component, an intracellular cellular component, or a combination thereof.

41. The method of any one of claims 1-40, wherein the plurality of cellular component binding reagents comprises a protein, an antibody, an aptamer, a tetramer, a protein scaffold, or a combination thereof, optionally wherein the aptamer and the reagent oligonucleotide is a single polynucleotide.

42. The method of any one of claims 1-41, wherein the plurality of cellular component binding reagents comprises at least 10 cellular component binding reagents.

43. The method of any one of claims 1-42, wherein the reagent oligonucleotide is attached, releasably attached, covalently attached, non-covalently attached, and/or conjugated to the cellular component binding reagent.

44. The method of any one of claims 1-43, wherein the reagent oligonucleotide is associated with the cellular component through a UV photocleavable group and/or a chemical labile group.

45. The method of any one of claims 1-44, wherein the reagent oligonucleotide is associated with the cellular component through a linker, optionally wherein the linker comprises a carbon chain, optionally wherein the carbon chain comprises 2-30 carbons, optionally wherein the carbon chain comprises 12 carbons, and optionally wherein the linker comprises 5′ amino modifier C12 (5AmMC12), or a derivative thereof.

46. The method of any one of claims 1-45, wherein the reagent oligonucleotide is 10 to 500 nucleotides in length, wherein the reagent-specific sequence is 5 to 495 nucleotides in length, and/or wherein the detection sequence is 5 to 495 nucleotides in length.

47. The method of any one of claims 1-46, wherein one or more of the reagent oligonucleotides each comprises two or more reagent-specific sequences and/or two or more detection sequences, and/or wherein one or more of the reagent oligonucleotides each has a hairpin structure.

48. The method of any one of claims 1-47, wherein the reagent oligonucleotides comprise an identical detection sequence, and/or wherein two of the reagent oligonucleotides comprise different detection sequences.

49. The method of any one of claims 1-48, comprising: amplifying the reagent oligonucleotides associated with or previously associated with the cellular component binding reagents bound to the cellular components of or from the cells of the sample to obtain amplified reagent oligonucleotides,
wherein contacting the reagent oligonucleotides, associated with or previously associated with the cellular component binding reagents bound to the cellular components of or from the cells of the sample, comprises: contacting the amplified reagent oligonucleotides with the plurality of solid supports, thereby obtaining amplified reagent oligonucleotides bound to the plurality of solid supports, and
wherein contacting the reagent oligonucleotides bound to the plurality of solid supports comprises: contacting the amplified reagent oligonucleotides bound to the plurality of solid supports with the detection oligonucleotide, thereby obtaining amplified reagent oligonucleotides bound to both the plurality of solid supports and the detection oligonucleotide.

50. The method of any one of claims 1-49, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences.

51. The method of any one of claims 1-50, wherein two solid supports of the plurality of solid supports comprise different quantities of the one or more first detectable moieties, and/or wherein two solid supports of the plurality of solid supports comprise different first detectable moieties.

52. The method of any one of claims 1-51, wherein all solid supports of the plurality of solid supports are distinguishable from each other by the presence and/or amount of the one or more first detectable moieties associated thereto.

53. The method of any one of claims 1-52, wherein the one or more first detectable moieties is attached, releasably attached, covalently attached, non-covalently attached, and/or conjugated to the solid support.

54. The method of any one of claims 1-53, wherein the plurality of solid supports comprises at least 10 solid supports.

55. The method of any one of claims 1-54, wherein the solid support comprises a bead.

56. The method of any one of claims 1-55, wherein the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo (dT) conjugated bead, a silica bead, a silica-like bead, an anti-biotin microbead, an anti-fluorochrome microbead, or any combination thereof.

57. The method of any one of claims 1-56, wherein the solid support comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof.

58. The method of any one of claims 1-57, wherein each of the plurality of the solid support oligonucleotides is 10 to 500 nucleotides in length, and/or wherein the capture sequence of each of the plurality of solid support oligonucleotides is 10 to 500 nucleotides in length.

59. The method of any one of claims 1-58, wherein the detection oligonucleotide is 10 to 500 nucleotides in length, and/or the binding sequence is 10 to 500 nucleotides in length.

60. The method of any one of claims 1-59, wherein the one or more second detectable moieties is attached, releasably attached, covalently attached, non-covalently attached, and/or conjugated to the detection oligonucleotide.

61. A kit comprising:
a plurality of cellular component binding reagents (1) capable of binding to different cellular components, or regions thereof, and (2) each associated with a reagent oligonucleotide comprising (i) a reagent-specific sequence specific to a cellular component binding reagent associated with the reagent oligonucleotide, and (ii) a detection sequence;
a plurality of solid supports, wherein each of the plurality of solid supports is associated with one or more first detectable moieties, or precursors thereof, and comprises a plurality of solid support oligonucleotides, and wherein different solid supports of the plurality of solid supports comprise different capture sequences for binding to different reagent-specific sequences of reagent oligonucleotides; and/or
a detection oligonucleotide associated with one or more second detectable moieties, or precursors thereof, and comprising a binding sequence capable of binding to the detection sequences of the reagent oligonucleotides.

62. The kit of claim 61, wherein one or more of the first detectable moieties and/or the second detectable moieties comprise an optical moiety, a luminescent moiety, an electrochemically active moiety, a nanoparticle, or a combination thereof.

63. The kit of any one of claims 61-62, wherein the luminescent moiety comprises a chemiluminescent moiety, an electroluminescent moiety, a photoluminescent moiety, or a combination thereof.

64. The kit of any one of claims 61-63, wherein the photoluminescent moiety comprises a fluorescent moiety, a phosphorescent moiety, or a combination thereof.

65. The kit of any one of claims 61-64, wherein the fluorescent moiety comprises a fluorescent dye.

66. The kit of any one of claims 61-65, wherein the nanoparticle comprises a quantum dot.

67. The kit of any one of claims 61-66, wherein each of the plurality of solid supports is associated with two distinct first detectable moieties, and wherein two solid supports of the plurality of solid supports comprise different types and/or quantities of the two distinct first detectable moieties.

68. The kit of any one of claims 61-67, wherein two of the plurality of cellular component binding reagents are capable of binding to two different cellular components, and/or wherein two of the plurality of cellular component binding reagents are capable of binding to two different regions of a cellular component.

69. The kit of any one of claims 61-68, wherein the cellular components comprise a protein, a lipid, a carbohydrate, or a combination thereof, and/or wherein the cellular components comprise an extracellular cellular component, a cell surface cellular component, an intracellular cellular component, or a combination thereof.

70. The kit of any one of claims 61-69, wherein the plurality of cellular component binding reagents comprises a protein, an antibody, an aptamer, a tetramer, a protein scaffold, or a combination thereof, optionally wherein the aptamer and the reagent oligonucleotide is a single polynucleotide.

71. The kit of any one of claims 61-70, wherein the plurality of cellular component binding reagents comprises at least 10 cellular component binding reagents.

72. The kit of any one of claims 61-71, wherein the reagent oligonucleotide is attached, releasably attached, covalently attached, non-covalently attached, and/or conjugated to the cellular component binding reagent.

73. The kit of any one of claims 61-72, wherein the reagent oligonucleotide is associated with the cellular component through a UV photocleavable group and/or a chemical labile group.

74. The kit of any one of claims 61-73, wherein the reagent oligonucleotide is associated with the cellular component through a linker, optionally wherein the linker comprises a carbon chain, optionally wherein the carbon chain comprises 2-30 carbons, optionally wherein the carbon chain comprises 12 carbons, and optionally wherein the linker comprises 5′ amino modifier C12 (5AmMC12), or a derivative thereof.

75. The kit of any one of claims 61-74, wherein the reagent oligonucleotide is 10 to 500 nucleotides in length, wherein the reagent-specific sequence is 5 to 495 nucleotides in length, and/or wherein the detection sequence is 5 to 495 nucleotides in length.

76. The kit of any one of claims 61-75, wherein one or more of the reagent oligonucleotides each comprises two or more reagent-specific sequences and/or two or more detection sequences, and/or wherein one or more of the reagent oligonucleotides each has a hairpin structure.

77. The kit of any one of claims 61-76, wherein the reagent oligonucleotides comprise an identical detection sequence, and/or wherein two of the reagent oligonucleotides comprise different detection sequences.

78. The kit of any one of claims 61-77, wherein at least two solid support oligonucleotides of a solid support of the plurality of solid supports comprises an identical capture sequence for binding to one of the reagent-specific sequences, and wherein a solid support oligonucleotide of a first solid support and a solid support oligonucleotide of a second solid support of the plurality of solid supports comprise different capture sequences for binding to two different reagent-specific sequences of the reagent-specific sequences.

79. The kit of any one of claims 61-78, wherein two solid supports of the plurality of solid supports comprise different quantities of the one or more first detectable moieties, and/or wherein two solid supports of the plurality of solid supports comprise different first detectable moieties.

80. The kit of any one of claims 61-79, wherein all solid supports of the plurality of solid supports are distinguishable from each other by the presence and/or amount of the one or more first detectable moieties associated thereto.

81. The kit of any one of claims 61-80, wherein the one or more first detectable moieties is attached, releasably attached, covalently attached, non-covalently attached, and/or conjugated to the solid support.

82. The kit of any one of claims 61-81, wherein the plurality of solid supports comprises at least 10 solid supports.

83. The kit of any one of claims 61-82, wherein the solid support comprises a bead.

84. The kit of any one of claims 61-83, wherein the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo (dT) conjugated bead, a silica bead, a silica-like bead, an anti-biotin microbead, an anti-fluorochrome microbead, or any combination thereof.

85. The kit of any one of claims 61-84, wherein the solid support comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof.

86. The kit of any one of claims 61-85, wherein each of the plurality of the solid support oligonucleotides is 10 to 500 nucleotides in length, and/or wherein the capture sequence of each of the plurality of solid support oligonucleotides is 10 to 500 nucleotides in length.

87. The kit of any one of claims 61-86, wherein the detection oligonucleotide is 10 to 500 nucleotides in length, and/or wherein the binding sequence is 10 to 500 nucleotides in length.

88. The kit of any one of claims 61-87, wherein the one or more second detectable moieties is attached, releasably attached, covalently attached, non-covalently attached, and/or conjugated to the detection oligonucleotide.
